(19) United States
(12) Patent Application Publication
(10) Pub. No.: US 2015/0079673 A1
(43) Pub. Date: Mar. 19, 2015

(54) HEPATOCYTES IN CO-CULTURE AND USES THEREOF

(71) Applicant: Colorado State University Research Foundation, Fort Collins, CO (US)

(72) Inventors: Salman R. Khetani, Fort Collins, CO (US); Michael D. Lehrer, Council Bluffs, IA (US)

(21) Appl. No.: 14/323,783

(22) Filed: Jul. 3, 2014

Related U.S. Application Data
(60) Provisional application No. 61/842,792, filed on Jul. 3, 2013.

(51) Int. Cl. C12N 5/071

(52) U.S. Cl. CPC C12N 5/067; C12N 2502/16
USPC 435/347

(57) ABSTRACT

The present disclosure provides a human hepatocyte co-culture which maintains the phenotype of cells from human donors with diabetes, and methods of using same. The hepatocyte co-culture system provides an in vitro model in which both cell viability and phenotype are maintained for extended periods relative to primary human hepatocyte monocultures, and is used in methods for high throughput screening and evaluation of drug candidates, and kits for performing such testing.

### REFERENCE TO RELATED APPLICATIONS

This application is a non-provisional application claiming priority to U.S. Provisional Application No. 61/842792 filed Jul. 3, 2013 and entitled “HEPATOCYTES IN CO-CULTURE AND USES THEREOF” which is hereby incorporated by reference in its entirety.

### FIELD OF THE INVENTION

The disclosure relates to in vitro cultures of human hepatic cells (hepatocytes), and in particular to co-culture systems including human hepatocytes, and their use in developing and screening drugs for disorders of the liver.

### BACKGROUND OF THE INVENTION

Reliable in vitro liver models are needed for the study of the liver's role in metabolic disorders which involve the liver, including Type 2 diabetes, metabolic syndrome, non-alcoholic fatty liver disease (NAFLD), non-alcoholic steatohepatitis (NASH) and cardiovascular disease. Cross-species variations in liver pathways can however be significant, and human in vitro liver models are therefore needed for the study of such diseases. Liver slices are used, but are limited by relatively short-term in vitro viability. Immortalized/cancerous cell lines are used, but are known to display an abnormal repertoire of liver function. Primary human hepatocytes (PHHs) are therefore commonly recognized as the “gold standard” of in vitro human liver models.

PHHs have an intact cell architecture and are relatively simple to use in vitro. While donor livers are in short supply, a single liver can supply a relatively high yield of PHHs (about 10-20 billion cells), and recent advances in cryopreservation allow for banking of PHH lots. It is not however always possible to culture cryopreserved hepatocytes in culture models and obtain the same functionality as observed with freshly PHHs. Furthermore, while conventional culture models commonly use extracellular matrix coatings or gels to support PHHs in culture, they do not provide other microenvironmental cues. These culture models typically display a precipitous (hours) decline in various measures of liver function, which limits their utility for accurately predicting the human in vivo response. For example, such models are highly limited in their glucose handling capacity and in responsiveness to hormones. Robust culture systems that can aid in investigation of the liver's role in Type 2 diabetes and other human metabolic diseases, and for discovering and screening novel drugs for such diseases are needed. Such systems should exhibit: reproducible compatibility with banked cryopreserved human cells; in vivo-like and long-term (weeks) glucose handling and hormonal responsiveness; and require minimal (<10 mg) drug quantities to demonstrate a response, given limitations on manufacturing scale-up of new drug candidates.

### SUMMARY OF THE INVENTION

In one aspect, the present disclosure provides a composition comprising a population of hepatocytes and at least one non-parenchymal cell population in co-culture, wherein the hepatocytes are obtained from one or more human donors suffering from a metabolic disorder of the liver. The metabolic disorder of the liver may be for example Type 2 diabetes mellitus. The composition may further comprise a culture substrate, and the population of hepatocytes may be disposed in a micropattern on the culture substrate. The micropattern may be a random or a predetermined micropattern on the culture substrate. The population of hepatocytes and the at least one non-parenchymal cell population may be maintained in vitro for at least three days, at least five days, at least one week, at least two weeks, at least three weeks, and as long as six weeks.

In any of the compositions and methods described, the hepatocytes may be primary human hepatocytes. Hepatocytes may be obtained from one or more human donors suffering from a metabolic disorder of the liver, such as but not limited to Type 2 diabetes mellitus. The population of hepatocytes may demonstrate, relative to donor hepatocytes obtained from non-diabetic donors, an increased rate of basal gluconeogenesis or hormone-induced gluconeogenesis. At least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. For example, stromal cells may comprise 3T3-J2 murine embryonic fibroblasts. The at least one non-parenchymal cell population may be obtained from an individual not suffering from a metabolic disorder of the liver, from an individual suffering from a metabolic disorder of the liver, or combinations thereof. The culture substrate may comprise a glass surface, a polystyrene surface, or a silicon surface. A biopolymer scaffold may be disposed on the culture substrate. The micropattern may be a random or a predetermined two-dimensional pattern of multiple microdots, defined by a microdot diameter and an edge-to-edge spacing between each of any two neighboring microdots. For example, when the micropattern comprises a predetermined two-dimensional pattern of multiple microdots, each microdot may have a diameter of 10 μm to 1000 μm, and the edge-to-edge spacing between each microdot may be at least about 200 μm to about 1000 μm. A cell adhesion molecule may be applied to the culture substrate at the microdots, the cell adhesion molecule being a material to which the hepatocytes selectively adhere relative to inter-microdot space to which the cell adhesion molecule is not applied. The non-parenchymal cell population may occupy the inter-microdot space which is not occupied by the hepatocytes. The cell adhesion molecule may be selected for example from collagen, fibronectin, vitronectin, laminin, Arg-Gly-Asp (RGD) peptide, Tyr-Ile-Gly-Ser-Arg (YIGSR) peptide, glycosaminoglycans, hyaluronic acid, integrins, ICAMs, selectins, cadherins and cell surface protein-specific antibodies.

In another aspect, the present disclosure provides a method for preserving the diabetic phenotype of a population of primary human hepatocytes in vitro following isolation of the diabetic hepatocytes from one or more human donors suffering from diabetes, the method comprising: co-culturing the population of diabetic hepatocytes with at least one non-parenchymal cell population, and maintaining the co-culture for at least 3 days. The method may comprise maintaining the co-culture for at least 5 days, at least one week, at least two weeks, at least three weeks, and as long as six weeks. The diabetic phenotype may be identified for example by at least one of: increased basal gluconeogenesis, stimulation of gluconeogenesis by hormones, and maintenance of cellular morphology, relative to donor hepatocytes obtained from non-diabetic donors. Co-culturing the population of diabetic hepatocytes with at least one non-parenchymal cell population may comprise co-culturing on a culture substrate, wherein the population of diabetic hepatocytes are disposed in a micropattern on the culture substrate. The method may further comprise forming the micropattern on the culture substrate by disposing a cell adhesion molecule on the culture substrate at the microdots, wherein the cell adhesion molecule is a material to which the diabetic hepatocytes selectively adhere relative to inter-microdot space. The method may further comprise seeding the diabetic hepatocytes onto the culture surface having the micropattern of cell adhesion molecule. The method may further comprise seeding the non-parenchymal cell population onto the culture surface such that the non-parenchymal cell population occupies inter-microdot space on the culture surface.

In another aspect, the present disclosure provides a method of determining the efficacy of a test compound as an therapeutic agent for treating a metabolic disorder of the liver, the method comprising: contacting a co-culture of a population of hepatocytes and at least one non-parenchymal cell population with the test compound, wherein the hepatocytes are obtained from one or more human donors suffering from a metabolic disorder of the liver; maintaining the co-culture for a time and under conditions sufficient to allow glucose production by the hepatocytes; and determining a level of glucose production by the hepatocytes, wherein the level of glucose production relative to the level of glucose production in a population of control hepatocytes is indicative of the efficacy of the test compound as an therapeutic agent. The hepatocytes may be obtained for example from one or more human donors suffering from Type 2 diabetes mellitus. The co-culture of the population of diabetic hepatocytes and at least one non-parenchymal cell population may comprise the population of hepatocytes disposed in a micropattern on a culture substrate. The micropattern may be a predetermined micropattern on the culture substrate. The method may further comprise, prior to determining the level of glucose production by the hepatocytes: depleting the co-culture of glycogen in glucose-free medium for a period of at least about twelve hours; contacting the co-culture with at least one substrate of a gluconeogenesis enzyme; and maintaining the co-culture for an additional period of at least about 12 hours under conditions sufficient for glucose production in the hepatocytes to occur. The co-culture may be maintained for a period of at least about 24 hours, at least about 48 hours, or at least about 72 hours under conditions sufficient for glucose production in the hepatocytes to occur. The at least one substrate of a gluconeogenesis enzyme may be for example lactate or pyruvate. The test compound may be a small molecule, a peptide, a polypeptide, an oligonucleotide, a polynucleotide, or an antibody.

In another aspect, the present disclosure provides a method of determining the efficacy of a test compound as a therapeutic agent for treating a metabolic disorder of the liver, the method comprising: contacting a co-culture of a population of hepatocytes and at least one non-parenchymal cell population with the test compound, wherein the hepatocytes are obtained from one or more human donors suffering from metabolic disorder of the liver; maintaining the co-culture for a time and under conditions sufficient to allow expression of at least one glucose metabolism protein or a nucleic acid sequence expressing a glucose metabolism protein; and determining a level of expression of the protein or nucleic acid sequence by the hepatocytes, wherein the expression level of the glucose metabolism protein, or a nucleic acid sequence expressing a glucose metabolism protein relative to the expression level of glucose metabolism protein, or a nucleic acid sequence expressing a glucose metabolism protein in a control cell is indicative of the efficacy of the test compound as a therapeutic agent for treating the metabolic disorder of the liver. The hepatocytes may be obtained for example from one or more human donors suffering from Type 2 diabetes mellitus. The glucose metabolism protein may be selected for example from: glycogen synthetase, glucose-6-phosphatase, glucose transporter 2 and phosphoenolpyruvate carboxykinase. Determining the level of expression of the glucose metabolism protein, or a nucleic acid sequence expressing a glucose metabolism protein may for example comprise performing a quantitative polymerase chain reaction, an ELISA assay, or a Western blot assay. The test compound may be a small molecule, a peptide, a polypeptide, an oligonucleotide, a polynucleotide, or an antibody.

In another aspect, the present disclosure provides a method of identifying an agent useful for modulating a biological activity of interest in a hepatocyte, the method comprising: contacting a co-culture of a population of hepatocytes and at least one non-parenchymal cell population with a candidate agent, wherein the hepatocytes are obtained from one or more human donors suffering from a metabolic disorder of the liver; maintaining the co-culture for a time and under conditions sufficient for the hepatocytes to generate a signal indicative of the biological activity; and detecting the signal generated by hepatocytes in the presence of the test agent, wherein the signal relative to a signal generated in a control cell is indicative of an effect on the biological activity of interest in the hepatocytes. The hepatocytes may be obtained for example from one or more human donors suffering from Type 2 diabetes mellitus. The signal indicative of the biological activity of interest may be for example a protein expression level or a protein secretion level. The biological activity of interest may be glucose metabolism. The biological activity of interest may be albumin secretion or urea synthesis. The agent may be a small molecule, a peptide, a polypeptide, an oligonucleotide, a polynucleotide, and an antibody. The method may further comprise determining the toxicity of the agent by measuring in the hepatocytes at least one cell signal indicative of cell toxicity. The at least one signal indicative of cell toxicity may be selected from: cellular necrosis, loss of membrane integrity, cell lysis, decrease in cell viability, apoptosis, mitochondrial membrane potential, mitochondrial DNA, ER stress, and steatosis.

In another aspect, the present disclosure provides a method for determining the cellular toxicity of a candidate therapeutic agent, the method comprising: contacting a co-culture of a population of hepatocytes and at least one non-parenchymal cell population with the candidate therapeutic agent, wherein the hepatocytes are obtained from one or more human donors suffering from a metabolic disorder of the liver; maintaining the co-culture for a time and under conditions sufficient for the hepatocytes to generate a signal indicative of cellular toxicity induced by the candidate therapeutic agent; and detecting the signal generated by hepatocytes in the presence of the candidate therapeutic agent, wherein the signal relative to a signal generated in a control cell is indicative of a toxic effect of the candidate agent on the hepatocytes. The hepatocytes may be obtained for example from one or more human donors suffering from Type 2 diabetes mellitus. The signal indicative of cell toxicity may be cellular necrosis, loss of membrane integrity, cell lysis, decrease in cell viability, apoptosis, mitochondrial membrane potential, mitochondrial DNA, ER stress, and steatosis.

In another aspect, the present disclosure provides a kit for determining the effect of a test agent on hepatocytes, the kit comprising: a co-culture of a population of hepatocytes and at least one non-parenchymal cell population, wherein the hepatocytes are obtained from one or more human donors suffering from a metabolic disorder of the liver. The hepatocytes may be obtained for example from one or more human donors suffering from Type 2 diabetes mellitus. The kit may further comprise a culture medium. The kit may further comprise a culture substrate having a surface and a cell adhesion molecule, the cell adhesion molecule disposed in a micropattern on the culture substrate. The micropattern may be a predetermined micropattern on the culture substrate. The population of hepatocytes may demonstrate, relative to donor hepatocytes obtained from non-diabetic donors, increased rate of basal gluconeogenesis. The kit may further comprise a label capable of generating a signal indicative of a level of a cellular activity of interest in the hepatocytes, such as but not limited a vital dye, a lipid dye, a colorimetric agent, or a bioluminescent marker.

In another aspect, the present disclosure provides a method for measuring effects on liver function of chronic changes in a level of at least one metabolite selected from glucose, fructose and fatty acids relative to a control level of the metabolite, the method comprising: contacting a co-culture of a population of hepatocytes and at least one non-parenchymal cell population with a predetermined amount of the metabolite or metabolites, wherein the hepatocytes are obtained from one or more human donors suffering from a metabolic disorder of the liver; maintaining the co-culture for a time and under conditions sufficient for the hepatocytes to generate a signal indicative of modified cellular function induced by the predetermined amount of the metabolite or metabolites; and detecting the signal generated by hepatocytes in the presence of the metabolite or metabolites, wherein the signal relative to a signal generated in a control cell subject to the same conditions is indicative of an effect of the amount of the metabolite or metabolites on the hepatocytes. In the method, the signal indicative of modified cellular function is selected from the group consisting of: modified transcription of a protein gene, modified translation a protein gene, modified secretion of a protein, cellular necrosis, loss of membrane integrity, cell lysis, decrease in cell viability, apoptosis, mitochondrial membrane potential, mitochondrial DNA, endoplasmic reticulum (ER) stress, and steatosis. The population of hepatocytes may be disposed in a predetermined micropattern on a culture substrate.

### BRIEF DESCRIPTION OF THE DRAWINGS

FIG. 1 is a schematic diagram of steps for establishing a micropatterned co-culture of primary human hepatocytes and stromal cells.

FIG. 2 is a bar graph of basal glucose production (μM) in human hepatocytes from non-diabetic (3 bars at left) and Type 2 diabetic human donors (2 bars at right), maintained in micropatterned co-culture with 3T3-J2 murine embryonic fibroblasts, showing higher basal rate of gluconeogenesis in diabetic compared to non-diabetic cells.

FIG. 3 is a bar graph of glucose production (μM) at 24 hours, in human hepatocytes maintained in micropatterned co-culture with 3T3-J2 murine embryonic fibroblasts, subjected to glycogen depletion for 12 hours followed by stimulation of gluconeogenesis using lactate or pyruvate, in the absence of hormones, or in the presence of insulin, glucagon or dexamethasone as indicated.

FIG. 4A is a bar graph comparing gluconeogenesis (μg glucose/well/24 hours) after 1 week in human hepatocytes maintained in micropatterned co-culture with 3T3-J2 murine embryonic fibroblasts in glucose free medium and in the presence or absence of insulin or glucagon (upper panel). FIG. 4B is a bar graph comparing gluconeogenesis (μg glucose/well/24 hours) after 1 week and in human hepatocytes maintained in conventional monoculture in the presence or absence of insulin and glucagon as indicated.

FIG. 5 is a graph of albumin secretion rate (μg/day/million cells) over time (days) in a population of pure (isolated) primary human hepatocytes in a conventional culture system (solid circles) and in a population of primary human hepatocytes in a micropatterned co-culture with 3T3-H2 murine embryonic fibroblast cells (open circles).

FIG. 6 is a graph of urea synthesis rate (μg/day/million cells) over time (days) in a population of pure (isolated) primary human hepatocytes in a conventional culture system (solid circles) and in a population of primary human hepatocytes in a micropatterned co-culture with 3T3-H2 murine embryonic fibroblast cells (open circles).

FIG. 7 is a bar graph comparing cumulative albumin secretion (μg; at left of graph) and urea synthesis (μg; at right of graph) over two weeks test populations of primary human hepatocytes (PHHs) in either a random co-culture, or in a micropatterned co-culture, with 3T3-H2 murine embryonic fibroblast cells, where varying diameters of the PHH islands and varying edge-to-edge distances among the islands were used, while total cell numbers/ratios were held constant. For example, “36/90” indicates a micropattern defined by 36 μm diameter islands, with 90 μm edge-to-edge spacing.

FIG. 8 is a graph of urea production in human hepatocytes maintained in micropatterned co-culture with 3T3-J2 murine embryonic fibroblasts, for a period of up to 8 days, in the presence of varying levels of glucose from 0 mM to 25 mM.

FIG. 9A is a graph of albumin secretion in human hepatocytes maintained in micropatterned co-culture with 3T3-J2 murine embryonic fibroblasts, in the presence of varying levels of glucose from 0 μM to 50 μM, showing down regulation of albumin secretion at high levels of glucose. FIG. 9B is a graph of glucose production at 24 hours, and following administration of a varying dose of metformin, in human hepatocytes maintained in micropatterned co-culture with 3T3-J2 murine embryonic fibroblasts, showing dose-dependent blockage by metformin of gluconeogenesis.

FIG. 10A is a photomicrograph of pure hepatocytes under conventional culture conditions after 1 day.

FIG. 10B is a photomicrograph of pure hepatocytes under conventional culture conditions after 1 week.

FIG. 10C is a photomicrograph of hepatocytes after 1 week in micropatterned co-culture with 3T3-J2 murine embryonic fibroblasts.

FIG. 10D is a photomicrograph of hepatocytes after 2 weeks in micropatterned co-culture with 3T3-J2 murine embryonic fibroblasts, showing transport of neurometric dye by transporters into the bile canaliculi between hepatocytes.

FIG. 11A is a panel of four photomicrographs of human hepatocytes maintained in micropatterned co-culture with 3T3-J2 murine embryonic fibroblasts, indicating glycogenolysis in the cells under four conditions, after 24 hrs of respective treatment: 25 mM glucose, glucose free, glucose free+100 nm glucagon, and glucose free+100 nm insulin.

FIG. 11B is a panel of four photomicrographs of human hepatocytes maintained in micropatterned co-culture with 3T3-J2 murine embryonic fibroblasts, indicating via PAS stain for intracellular glycogen, glycogen synthesis in the cells under four conditions: 5 mM glucose, 12.5 mM glucose, 5 mM glucose+100 nm insulin, and 5 mM glucose+100 nm glucagon.

FIG. 12 is bar graph comparing CYP3A4 activity in hepatocytes from two human donors, maintained in micropatterned co-culture with 3T3-J2 murine embryonic fibroblasts, over a period of 0-21 days.

FIG. 13 is a bar graph of expression levels, as RT-qPCR levels normalized to expression level in suspension hepatocytes, of glycogen synthase (GYS), glucose-6-phosphatase (G6Pase), and glucose transporter 2 (SLC1A2), following 1 or 2 weeks in micropatterned co-culture with 3T3-J2 murine embryonic fibroblasts, or 3 days or 1 week in conventional (mono) culture.

FIG. 14A is a bar graph of expression level, as RT-qPCR level normalized to expression level in suspension hepatocytes, of phosphoenolpyruvate carboxykinase 1 (PCK1), following 1 or 2 weeks in micropatterned co-culture with 3T3-J2 murine embryonic fibroblasts, or 3 days or 1 week in conventional (mono) culture. FIG. 14B is a graph of glucose production in human hepatocytes maintained in micropatterned co-culture with 3T3-J2 murine embryonic fibroblasts for 4 hours and following a 3′-MPA dose (μM).

FIGS. 15A-15C show the effects of glucose concentration on MPCCs. FIG. 15A is a bar graph of lipid accumulation quantified by nile red staining after 6 days of glucose treatment at the concentrations indicated. FIG. 15B is a bar graph of urea and albumin production at day 6 of glucose treatment. FIG. 15C depicts micrographs of representative MPCC islands stained with nile red after 6 days of glucose treatment at, from left to right, 5 mM, 25 mM, 50 mM and 100 mM glucose concentrations. Error bars represent SEM, n=9 for nile red pixel intensity, n=3 for urea and albumin production at the concentrations indicated.

FIG. 16 is a bar graph of gluconeogenesis rate (nmol/hour/million cells) in human hepatocytes maintained in micropatterned co-culture with 3T3-J2 murine embryonic fibroblasts, under varying conditions and at varying time periods including re-use of the co-culture. “2 week reuse” refers to co-cultures probed for gluconeogenesis and hormonal responsiveness (with glucagon or insulin) on week 1 and then reused on week 2. “3 week reuse” refers to co-cultures probed for gluconeogenesis on week 2, and reused on week 3. Bars labeled “1 Week”, “2 week” or “3 week” indicate data from co-cultures probed for gluconeogenesis for the first time at the time point indicated.

FIG. 17A is a photomicrograph showing morphology of hepatocytes in MPCC (representative image from day 7).

FIG. 17B is a graph of rate of urea synthesis and albumin production over 3 weeks in MPCC.

FIG. 17C is a photomicrograph showing morphology of hepatocytes in monolayer culture (representative image from day 7).

FIG. 17D is a graph of rate of urea synthesis and albumin production over 7 days in monolayer culture.

### DETAILED DESCRIPTION OF THE DISCLOSURE

The present disclosure describes compositions and methods based in part on the surprising finding that primary human hepatocytes obtained from human donors suffering from Type 2 diabetes, when maintained in co-culture with nonparenchymal cells, such as for example fibroblast or fibroblast-derived cells, exhibit a higher basal level of gluconeogenesis than cells obtained from non-diabetic donors and maintained under the same conditions. Additionally, the hepatocytes in co-culture remain viable for extended periods relative to hepatocytes maintained in conventional (mono) culture, while at the same time also appearing to maintain phenotype for a period of at least about 3 days and as long as 6 weeks.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities and plural terms shall include the singular. For example, reference to “a cellular island” includes a plurality of such cellular islands and reference to “the cell” includes reference to one or more cells known to those skilled in the art, and so forth.

The use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present disclosure are generally performed according to conventional methods well known in the art and as described in various general and more specific references that are cited and discussed throughout the present specification unless otherwise indicated. Enzymatic and staining reactions, and purification techniques are performed according to manufacturer's specifications and protocols, as commonly accomplished in the art or as described herein. The nomenclatures used in connection with, and the laboratory procedures and techniques of, analytical chemistry, synthetic organic chemistry, and medicinal and pharmaceutical chemistry described herein are also those well known and commonly used in the art. Standard techniques are used for chemical syntheses, chemical analyses, pharmaceutical preparation, formulation, and delivery, and treatment of patients. An “immunogenic agent” or “immunogen” is capable of inducing an immunological response against itself on administration to a mammal, optionally in conjunction with an adjuvant.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “donor” includes human and other mammalian subjects from which primary cells such as primary hepatocytes may be obtained.

The term “patient” includes human and other mammalian subjects that receive either prophylactic or therapeutic treatment.

That the present disclosure may be more readily understood, select terms and phrases as used herein are defined below.

### A. CO-CULTURES OF PRIMARY HUMAN HEPATOCYTES & KITS

The present disclosure involves the use of co-cultures, i.e., parenchymal cell/non-parenchymal cell co-cultures. The co-cultures may be random co-cultures. Alternatively, the co-cultures may be micropatterned co-cultures (“MPCCs”). The parenchymal cells are human hepatocytes obtained from a mammalian donor suffering from a metabolic disorder of the liver, and the non-parenchymal cells are preferably fibroblast or fibroblast-derived cells. Metabolic disorders of the liver include but are not limited to Type 2 diabetes, metabolic syndrome, non-alcoholic fatty liver disease (NAFLD), non-alcoholic steatohepatitis (NASH) and cardiovascular disease. A non-limiting, exemplary co-culture is one which includes hepatocytes obtained from one or more human donors suffering from diabetes mellitus, such as Type 2 diabetes mellitus. At least one of the non-parenchymal cell populations may comprise stromal cell, such as but not limited to fibroblasts, fibroblast-derived cells, macrophages, endothelial cells, stellate cells, pericytes, inflammatory cells, cholangiocytes and other types of stromal cells, and combinations thereof. Fibroblasts may be for example mammalian fibroblasts, such as for murine embryonic fibroblasts. Non-limiting, exemplary fibroblasts are 3T3-J2 murine embryonic fibroblasts.

Co-culturing methods and techniques in general have been described in the literature, and in particular MPCC co-culturing materials, methods and techniques are described detail in Khetani and Bhatia, Nature Biotechnology, 2008, 26(1):120-126, the entire disclosure of which is incorporated herein by reference.

The present disclosure encompasses a composition comprising the combination of a population of primary mammalian hepatocytes and at least one non-parenchymal cell population in co-culture, wherein the hepatocytes are obtained from a donor suffering from a metabolic disorder of the liver. For example, the hepatocytes may be obtained from one or more human donors suffering from Type 2 diabetes mellitus. The co-cultures as described herein provide a useful in vitro model of metabolic disorders of the liver, such as Type 2 diabetes. Co-cultures of hepatocytes obtained from one or more human donors suffering from diabetes are clearly distinguished by a higher basal rate of gluconeogenesis than that observed in non-diabetic hepatocytes under the same conditions. The co-cultures of hepatocytes described herein thus also provide a unique platform for the development and testing of therapeutic agents for the treatment of metabolic disorders of the liver, including high-throughput screening of drug candidates for efficacy and toxicity.

In the compositions, at least one of the non-parenchymal cell populations may comprise stromal cells. Stromal cells may comprise fibroblasts, fibroblast-derived cells, macrophages, endothelial cells, stellate cells, pericytes, inflammatory cells, cholangiocytes and other types of stromal cells, and combinations thereof. Stromal cells may comprise 3T3-J2 murine embryonic fibroblasts.

The culture substrate may comprise a glass or elastomeric structure with a suitable culture surface, such as a glass, polystyrene, or silicon slide, or polystyrene dish, slide or microwells. A biopolymer scaffold may optionally be disposed on the culture substrate to further support and promote cell viability. Biopolymers suitable as scaffold material include but are not limited to alginate, chitosan, hyaluronate, fibrous proteins, collagen, silk and elastin.

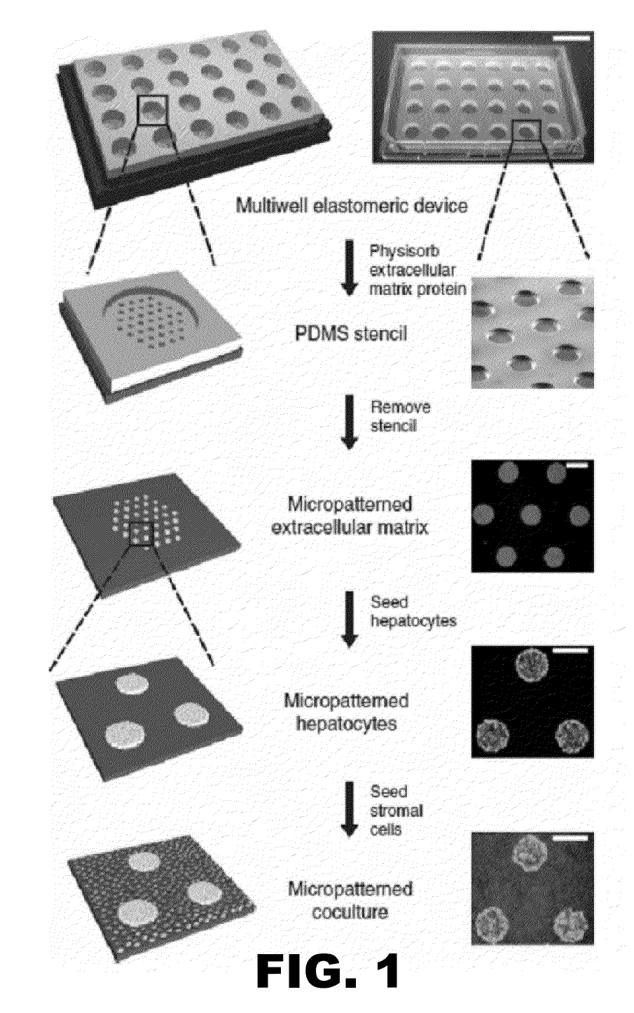

The co-cultures described herein encompass randomly distributed co-cultures of hepatocytes and non-parenchymal cells, such as stromal cells. As illustrated in part in FIG. 1, the co-cultures may alternatively be established according to a micropattern established on the culture surface. The micropattern may comprise for example a predetermined two-dimensional pattern of multiple microdots (“islands”) of the hepatocytes, wherein each microdot has approximately the same microdot diameter and each of any two neighboring microdots shares approximately the same edge-to-edge spacing. While the microdot diameters and microdot spacing may be varied, a micropattern characterized by microdots each having a diameter of 10 μm to 1000 μm, and an edge-to-edge spacing between each microdot of at least 200 μm to about 1000 μm, preferably a micropattern characterized by microdots each having a diameter of 300 μm to 700 μm, and an edge-to-edge spacing between each microdot of at least 200 μm to about 700 μm has been found to result in hepatocyte co-cultures that remain viable and show evidence of phenotype retention for several weeks.

In various aspects, each microdot may have a diameter of 10 μm to 100 μm, a diameter of 50 μm to 150 μm, a diameter of 100 μm to 200 μm, a diameter of 150 μm to 250 μm, a diameter of 200 μm to 300 μm, a diameter of 250 μm to 350 μm, a diameter of 300 μm to 400 μm, a diameter of 350 μm to 450 μm, a diameter of 400 μm to 500 μm, a diameter of 450 μm to 550 μm, a diameter of 500 μm to 600 μm, a diameter of 550 μm to 650 μm, a diameter of 600 μm to 700 μm, a diameter of 650 μm to 750 μm, a diameter of 700 μm to 800 μm, a diameter of 750 μm to 850 μm, a diameter of 800 μm to 900 μm, a diameter of 850 μm to 950 μm, and a diameter of 900 μm to 1000 μm,

In various aspects, the microdots may have an edge-to-edge spacing of 200 μm to 300 μm, an edge-to-edge spacing of 250 μm to 350 μm, an edge-to-edge spacing of 300 μm to 400 μm, an edge-to-edge spacing of 350 μm to 450 μm, an edge-to-edge spacing of 400 μm to 500 μm, an edge-to-edge spacing of 450 μm to 550 μm, an edge-to-edge spacing of 500 μm to 600 μm, an edge-to-edge spacing of 550 μm to 650 μm, an edge-to-edge spacing of 600 μm to 700 μm, an edge-to-edge spacing of 650 μm to 750 μm, an edge-to-edge spacing of 700 μm to 800 μm, an edge-to-edge spacing of 750 μm to 850 μm, an edge-to-edge spacing of 800 μm to 900 μm, an edge-to-edge spacing of 850 μm to 950 μm, and an edge-to-edge spacing of 900 μm to 1000 μm,

To establish the micropattern, a cell adhesion molecule may be applied to the culture substrate at the microdots, using for example a soft lithography technique as known in the art, using an elastomeric stamp, mold, stencil or conformable photomask. For example, a PDMS (polydimethylsiloxane) stencil can be used. The cell adhesion molecule is any molecule to which the hepatocytes selectively adhere relative to inter-microdot space, such as collagen, fibronectin, vitronectin, laminin, extracellular matrix proteins, Arg-Gly-Asp (RGD) peptide, Tyr-Ile-Gly-Ser-Arg (YIGSR) peptide, glycosaminoglycans, hyaluronic acid, integrins, ICAMs, selectins, cadherins and cell surface protein-specific antibodies. In one micropatterned hepatocyte co-culture, the cell adhesion molecule is for example any of the many extracellular matrix protein products available from a variety of commercial suppliers. Following seeding of the hepatocytes onto the micropattern, the non-parenchymal cell population may be seeded onto the culture surface to occupy the inter-microdot space which is not occupied by the hepatocytes.

The present disclosure also provides a kit for determining the effect of a test agent on hepatocytes, the kit comprising: a population of hepatocytes and at least one non-parenchymal cell population, wherein the hepatocytes are obtained from one or more human donors suffering a metabolic disorder of the liver. For example, the hepatocytes may be obtained from one or more human donors suffering from Type 2 diabetes mellitus. The kit may further comprise a culture medium and/or additional materials or reagents for testing various biological activities of the cells in culture. For example, the kit may contain separately packaged amounts of a glucose-free medium, pyruvate, lactate, glucose, insulin, glucagon, dexamethasone, metformin, a stain or dye such as but not limited to a fluorometric dye, a lipid dye such as Nile red, and/or a cellular stain for glycogen such as PAS stain. The kit may further comprise one or more culture substrates such as a glass, silicon, or polystyrene slide or culture well, and an amount of a cell adhesion molecule. The cell adhesion molecule may be disposed according to a micropattern on the culture substrate as described herein above. Alternatively the kit may provide an amount of the cell adhesion molecule and a PDMS stencil which can be used together to establish the micropattern on the culture substrate.

The kit may further comprise a reporter molecule or label capable of generating a signal indicative of a level of a cellular activity of interest in the hepatocytes, such as but not limited a vital dye, a lipid dye, a colorimetric agent, or a bioluminescent marker. The kit may include a detectable label such as a fluorophore, a radioactive moiety, an enzyme, a chromophore, a chemiluminescent label, or the like, and/or reagents for carrying out detectable labeling. The labels and/or reporter molecules, any calibrators and/or controls can be provided in separate containers or pre-dispensed into an appropriate assay format, for example, into microtiter plates.

It is contemplated for example that one or more of the presently disclosed co-cultures can be provided in the form of a kit with one or more containers such as vials or bottles, with each container containing a separate population of cells and/or reagents and washing reagents employed in an assay. The kit can comprise at least one container for conducting the assay, and/or a buffer, such as an assay buffer or a wash buffer, either one of which can be provided as a concentrated solution, a substrate solution for the detectable or a stop solution. Preferably, the kit comprises all components, i.e., reagents, standards, buffers, diluents, etc., which are necessary to perform the assay. The kit may contain instructions for determining the presence or amount of any metabolite, biomarker, label or reporter of interest in the co-culture, in paper form or computer-readable form, such as a disk, CD, DVD, or the like, and/or may be made available online.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate, vial, tube, and the like, or any combination thereof. Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

### B. USES OF MICRO-PATTERNED CO-CULTURES OF PRIMARY HUMAN HEPATOCYTES

Micro-patterned co-cultures of primary human hepatocytes as described herein may be used in various methods, such as but not limited to drug discovery and drug screening. For example, such co-culture systems can be used to develop and screen drug candidates for modulating hepatic glucose metabolism. Additionally, because human hepatocytes in MPCC demonstrate glucose toxicity, they can be used to study the effects of chronically elevated or reduced levels of glucose, fructose and fatty acids on the liver and liver function.

### Screening Assays

The present disclosure provides an in vitro model of metabolic diseases of the liver, including Type 2 diabetes, which can be utilized in various methods for identifying and screening of potential therapeutic agents, and for drug development. The compositions of the present disclosure exhibit a higher basal rate of gluconeogenesis than primary human hepatocytes obtained from nondiabetic donors, and thus the co-cultures described herein provide a useful platform for screening of anti-diabetic agents and/or agents for modulating liver function in other diseases of the liver.

The compositions of the present disclosure may be used in vitro to screen a wide variety of compounds, such as small molecules, antibodies, peptides, polypeptides, nucleic acid-based agents and the like, to identify agents having a therapeutic effect on liver function in a metabolic disorder of the liver. For example, various cellular functions in the hepatocytes may be assessed by observation of a level of a metabolite, a reporter molecule, a label, or gene expression level such as through gene fluorescence in the cell or in the culture media.

Gluconeogenesis and other liver functions such as albumin secretion, urea production, glycolysis and glycogen storage may be monitored in the presence and absence of one or more stimuli, and/or test compounds or agents (also referred to herein and in the claims as “candidate agents”). For example, hepatocytes in co-culture as described herein may be tested for any one or more of gluconeogenesis, albumin secretion, urea production, glycolysis and glycogen storage in the presence and absence of varying levels of glucose, insulin, glucagon, dexamethasone, and/or one or more stimuli, test compounds or agents.

Levels of biomarkers such as for example specific metabolites may also be used in screening assays for agents. This may also be accomplished using standard cytological and/or histological techniques including the use of immunocytochemical techniques employing stains that recognize specific cellular components such as lipids, or antibodies that specifically bind to biomarkers with antigenic activity.

In this case, stable micropattern cultures may be exposed to a test agent. After incubation, the micropattern cultures may be examined for change in biomarker production as an indication of the efficacy of the test substance. Varying concentrations of the drug may be tested to derive a dose-response curve.

### Target Validation

The compositions of the invention are particularly suited for use in target identification and target validation. The cultures and/or systems are useful for identifying targets and predicting the role of one or more biomolecules in liver function in a metabolic disorder of the liver. For example, the cultures and systems may be used to identify proteins playing a potential role in diabetic processes or diabetic liver pathways. Identified proteins may be modulated (e.g., up-regulated or down-regulated) in the co-cultures described herein, and processes and pathways related to diabetes may be assayed following modulation.

The cultures and/or systems are also useful for validating the predicted role of one or more biomolecules in liver function in a metabolic disorder of the liver. For example, proteins identified in preliminary studies (e.g., studies of primary hepatocytes in conventional culture systems or cryogenically preserved hepatocytes, studies in non-diabetic liver models, differential expression studies, etc.) as playing a potential role in diabetic processes or diabetic liver pathways can be tested in a composition as described herein to confirm the potential role. Proteins identified from preliminary studies, for example proteins suspected to play a role in diabetes or diabetic liver function, may be modulated (e.g., up-regulated or down-regulated) in the co-cultures described herein, and processes and pathways related to diabetes may be assayed following modulation. For example, candidate proteins can be “knocked out/down” using gene knockout or suppression techniques, for example, using various genomic editing techniques, or the introduction of RNA interference (RNAi) agents. Inhibition of liver pathways may be tested following down-regulation and candidate proteins thought to be important in diabetic liver function may be thus validated.

The present disclosure thus also encompasses various methods of using the hepatocyte co-cultures described herein. For example, the present disclosure also provides a method for preserving the phenotype of a population of primary human hepatocytes in vitro following isolation of the hepatocytes from one or more human donors suffering from a metabolic disorder of the liver, the method comprising: co-culturing the population of hepatocytes with at least one non-parenchymal cell population, and preserving the phenotype of the population of primary human hepatocytes in co-culture for at least 3 days. The method may comprise preserving the phenotype of the population of primary human hepatocytes in co-culture for at least 5 days, or at least two months. The phenotype may be identified for example by at least one of: increased basal gluconeogenesis relative to donor hepatocytes obtained from non-diabetic donors, stimulation of gluconeogenesis by hormones, and cellular morphology. The co-cultures may be MPCC co-cultures as described herein above.

The present disclosure also provides a method of determining the efficacy of a test compound as a therapeutic agent for treating a metabolic disorder of the liver, the method comprising: contacting a co-culture of a population of hepatocytes and at least one non-parenchymal cell population with the test compound, wherein the hepatocytes are obtained from one or more human donors suffering from a metabolic disorder of the liver; maintaining the co-culture for a time and under conditions sufficient to allow glucose production by the hepatocytes; and determining a level of glucose production by the hepatocytes, wherein the level of glucose production relative to the level of glucose production in a population of control hepatocytes is indicative of the efficacy of the test compound as an therapeutic agent for treating a metabolic disorder of the liver. The metabolic disorder of the liver may be for example Type 2 diabetes. The co-culture may be an MPCC co-culture as described elsewhere herein. The method may further comprise, prior to determining the level of glucose production by the hepatocytes: depleting the co-culture of glycogen in glucose-free medium for a period of at least about twelve hours; contacting the co-culture with at least one substrate of a gluconeogenesis enzyme; and maintaining the co-culture for a period of at least about 12 hours under conditions sufficient for glucose production in the hepatocytes to occur. The co-culture may be maintained for a period of at least about 24 hours, or at least 48 hours under conditions sufficient for glucose production in the hepatocytes to occur. The at least one substrate of a gluconeogenesis enzyme may be for example lactate or pyruvate. The test compound may be a small molecule, a peptide, a polypeptide, an oligonucleotide, a polynucleotide, or an antibody.

The present disclosure also provides a method of determining the efficacy of a test compound as a therapeutic agent for treating a metabolic disorder of the liver, the method comprising: contacting a co-culture of a population of hepatocytes and at least one non-parenchymal cell population with the test compound, wherein the hepatocytes are obtained from one or more human donors suffering from a metabolic disorder of the liver; maintaining the co-culture for a time and under conditions sufficient to allow expression of at least one glucose metabolism protein or a nucleic acid sequence expressing a glucose metabolism protein; and determining a level of expression of the glucose metabolism protein or a nucleic acid sequence expressing a glucose metabolism protein by the hepatocytes, wherein the expression level of the glucose metabolism protein or a nucleic acid sequence expressing a glucose metabolism protein relative to the expression level of glucose metabolism protein in a control cell is indicative of the efficacy of the test compound. The metabolic disorder of the liver may be for example Type 2 diabetes. The glucose metabolism protein may be selected for example from: glycogen synthetase, glucose-6-phosphatase, glucose transporter 2 and phosphoenolpyruvate carboxykinase. Determining the level of expression of the glucose metabolism protein or a nucleic acid sequence expressing a glucose metabolism protein may for example comprise performing a quantitative polymerase chain reaction, an ELISA assay, or a Western blot assay. The test compound may be a small molecule, a peptide, a polypeptide, an oligonucleotide, a polynucleotide, or an antibody.

The present disclosure also provides a method of identifying an agent useful for modulating a biological activity of interest in a hepatocyte, the method comprising: contacting a co-culture of a population of hepatocytes and at least one non-parenchymal cell population with a candidate agent, wherein the hepatocytes are obtained from one or more human donors suffering from a metabolic disorder of the liver; maintaining the co-culture for a time and under conditions sufficient for the hepatocytes to generate a signal indicative of the biological activity; and detecting the signal generated by hepatocytes in the presence of the test agent, wherein the signal relative to a signal generated in a control cell is indicative of an effect on the biological activity of interest in the hepatocytes. The metabolic disorder of the liver may be for example Type 2 diabetes. The signal indicative of the biological activity of interest may be for example a protein expression level or a protein secretion level. The biological activity of interest may be glucose metabolism. The biological activity of interest may be albumin secretion or urea synthesis. The agent may be a small molecule, a peptide, a polypeptide, an oligonucleotide, a polynucleotide, and an antibody.

Any of the methods may involve determining a baseline value, for example, of liver function such as gluconeogenesis, glycolysis, glycogen storage, enzyme activity, albumin secretion, urea production, and the like, in hepatocytes in co-culture before administering a dosage of a test agent, and comparing this with a value or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value does not change significantly, or decreases, a negative treatment outcome is indicated.

A control value (i.e., a mean and standard deviation) or profile may be determined for a control population. Typically the individuals in the control population have not received prior treatment. Measured values of the level or profile in a patient after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

### Toxicity Studies

In addition to the above-described uses of the cultures and/or systems of the invention in screening for therapeutic agents for treating a metabolic disorder of the liver, the co-cultures may also be used in toxicology studies to determine the toxicity of an agent identified as a potential therapeutic agent. Toxicology studies may be performed on co-cultures featuring hepatocytes from human donors suffering from a metabolic disorder of the liver, as described herein, which may be contrasted with comparable studies in cells from a different source. The co-cultures described herein may be used in vitro to test a variety of potential therapeutic compounds for their ability to cause cytotoxicity and/or cell death. Any of the screening methods described herein above may further comprise determining the toxicity of the agent by measuring in the hepatocytes at least one cell signal indicative of cell toxicity.

Toxicity results may be assessed for example by observation of cellular necrosis, loss of membrane integrity, cell lysis, decrease in cell viability, apoptosis, mitochondrial membrane potential, mitochondrial DNA, ER stress, and steatosis, using any one or more of vital staining techniques, ELISA assays, RT-qPCR, immunohistochemistry, and the like or by analyzing the cellular content of the culture, e.g., by total cell counts, and differential cell counts or by metabolic markers such as MTT and XTT.

In one aspect of the invention, a stable, growing co-culture is established having a desired size (e.g., island size and distance between islands), morphology and may also include a desired oxygen gradient. The cells in the co-culture are exposed to varying concentrations of a test agent. After incubation with a test agent, the culture is examined to determine the highest tolerated dose, i.e., the concentration of test agent at which the earliest morphological abnormalities appear or are detected. Cytotoxicity testing may also be performed using a variety of supravital dyes to assess cell viability in the culture system, using techniques known to those skilled in the art. Once a testing range is established, varying concentrations of the test agent can be examined for their cytotoxic effect.

The present disclosure thus also provides a method for determining the cellular toxicity of a candidate therapeutic agent, the method comprising: contacting a co-culture of a population of hepatocytes and at least one non-parenchymal cell population with the candidate therapeutic agent, wherein the hepatocytes are obtained from one or more human donors suffering from a metabolic disorder of the liver; maintaining the co-culture for a time and under conditions sufficient for the hepatocytes to generate a signal indicative of cellular toxicity induced by the candidate therapeutic agent; and detecting the signal generated by hepatocytes in the presence of the candidate therapeutic agent, wherein the signal relative to a signal generated in a control cell is indicative of a toxic effect of the candidate agent on the hepatocytes. The signal indicative of cell toxicity may be cellular necrosis, loss of membrane integrity, cell lysis, decrease in cell viability, apoptosis, mitochondrial membrane potential, mitochondrial DNA, ER stress, and steatosis.

Additionally, the present disclosure thus also provides a method for determining the effects of chronically elevated or reduced levels of glucose, fructose and/or fatty acids on the liver and liver function. The method comprises for example contacting a co-culture of a population of hepatocytes and at least one non-parenchymal cell population with a predetermined amount of one or more metabolites such as glucose, fructose, and or fatty acids, wherein the hepatocytes are obtained from one or more human donors suffering from a metabolic disorder of the liver; maintaining the co-culture for a time and under conditions sufficient for the hepatocytes to generate a signal indicative of modified cellular function induced by the predetermined amount of one or more metabolites; and detecting the signal generated by hepatocytes in the presence of the one or more metabolites, wherein the signal relative to a signal generated in a control cell subject to the same conditions is indicative of an effect of the amount of the one or more metabolites on the hepatocytes. The signal indicative of an effect on cell function may be a change in transcription, translation or secretion of a protein, cellular necrosis, loss of membrane integrity, cell lysis, decrease in cell viability, apoptosis, mitochondrial membrane potential, mitochondrial DNA, ER stress, and steatosis. The predetermined amount may be an amount which is elevated or reduced relative to a control amount which is representative of an amount of each metabolite which is considered within the range of normal in vivo values for the metabolite. The time over which the hepatocytes are exposed to the elevated or reduced level(s) of metabolite(s) may be, according to knowledge available to those of skill in the art, a period of days, weeks or months representing chronic elevation or reduction of the metabolite in a clinical population.

It will be readily apparent to those skilled in the art that other suitable modifications and adaptations of the methods of the disclosure described herein are contemplated and may be made using suitable equivalents without departing from the scope of the disclosure or the embodiments disclosed herein. Having now described the present disclosure in detail, the same will be more clearly understood by reference to the following examples, which are included for purposes of illustration only and are not intended to be limiting of the disclosure.

### C. EXAMPLES

The following examples are provided for exemplary guidance to make and use is the compounds and supplements according to the inventive subject matter. However, it should be recognized that numerous modifications may be made without departing from the inventive concept presented herein.

### Example 1

### Gluconeogenesis

Micropatterned co-cultures (MPCC cultures) of primary human hepatocytes and 3T3-J2 murine embryonic fibroblasts were prepared substantially as described in Khetani and Bhatia, Nature Biotechnology, 2008, 26(1):120-126 (the entire disclosure of which is incorporated herein by reference), except that some of the hepatocytes were obtained from human donors suffering from Type 2 diabetes, and cells were subsequently subjected to glucose-free medium to achieve glycogen depletion before being tested for gluconeogenesis. Hepatocytes were also obtained from non-diabetic donors.

Diabetic hepatocytes and non-diabetic hepatocyte populations were used to establish separate MPCC co-cultures using soft-lithographic techniques according to published protocol. Hepatocytes were maintained on polystyrene in islands (microdots) having a diameter of 500 micron islands, spaced 700 micron edge-to-edge. FIG. 1 is a schematic illustration of the protocol for establishing a micropatterned co-culture of primary hepatocytes and stromal cells on a multiwall elastomeric (polystyrene) device. Extracellular matrix protein was used as the cell adhesion molecule and a PDMS stencil used to establish the micropattern consisting of multiple, evenly spaced islands or microdots.

Conventional cultures (monocultures) of diabetic hepatocytes show a drastic decline in gluconeogenesis and little to no hormonal responsiveness for glucose production. To test for gluconeogenesis in diabetic hepatocytes in co-culture, the MPCC hepatocyte cultures comprising diabetic versus non-diabetic cells were subjected to glucose-free medium for 24-48 hours, thereby depleting the resident glycogen stores so that cells were only able to perform gluconeogenesis in the presence of the glucose precursors lactate and pyruvate. Gluconeogenesis rate was measured by incubating glycogen-depleted cultures with the substrates pyruvate and lactate, in glucose-free medium and glucose measurement in medium (Amplex Red) at 24 hours, thereby isolating the contribution of gluconeogenesis to glucose production, from potential residual glucose output due to glycogen depletion.

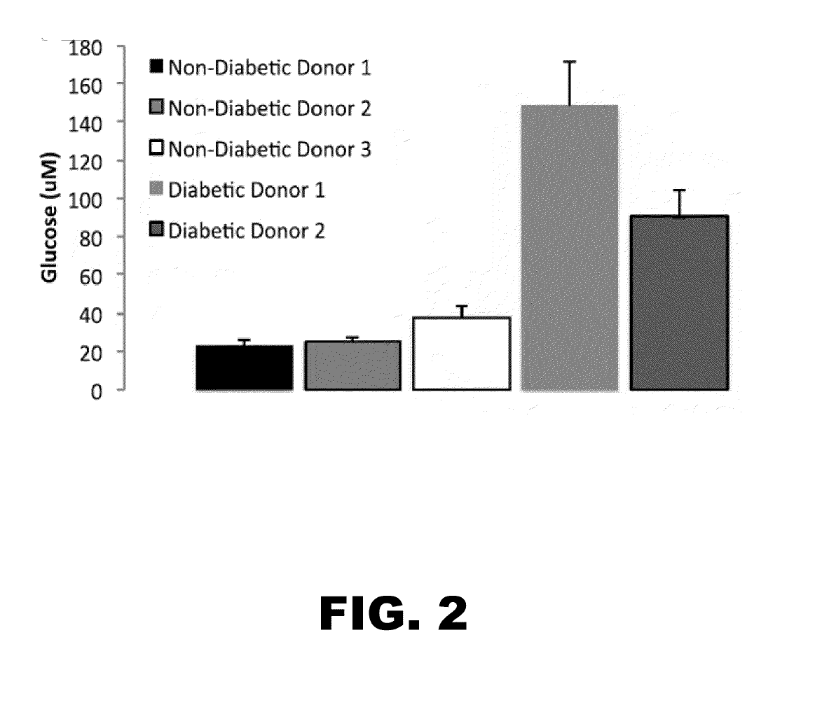

FIG. 2 is a bar graph of basal glucose production (μM) in human hepatocytes from non-diabetic (3 bars at left) and Type 2 diabetic human donors (2 bars at right), maintained in micropatterned co-culture with 3T3-J2 murine embryonic fibroblasts, showing higher basal rate of gluconeogenesis in diabetic compared to non-diabetic cells. Surprisingly, as shown in FIG. 2, the diabetic hepatocytes showed a higher rate of basal gluconeogenesis as compared to cells obtained from non-diabetic donors. This difference was not only significant, but also sustained for 48 hours (not shown).

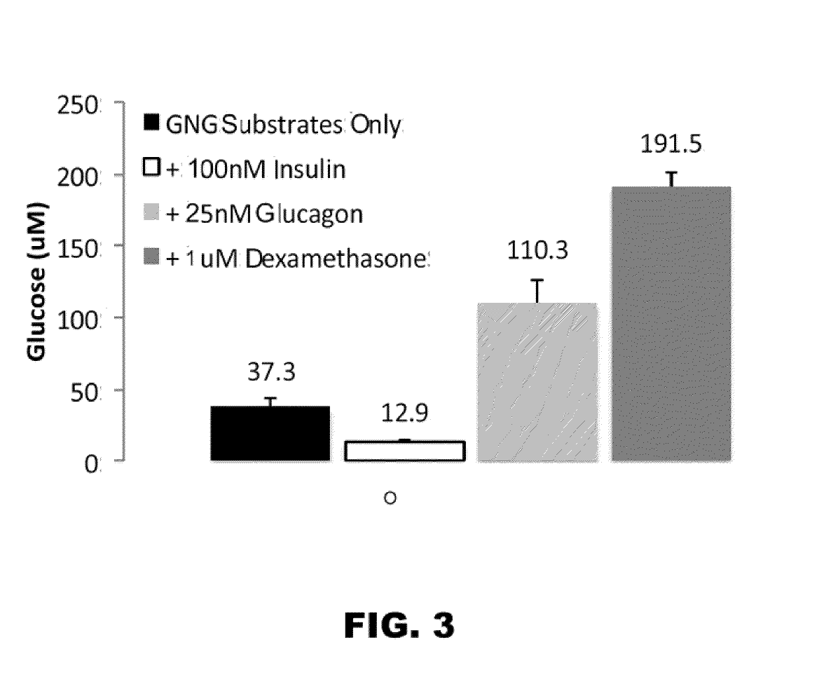

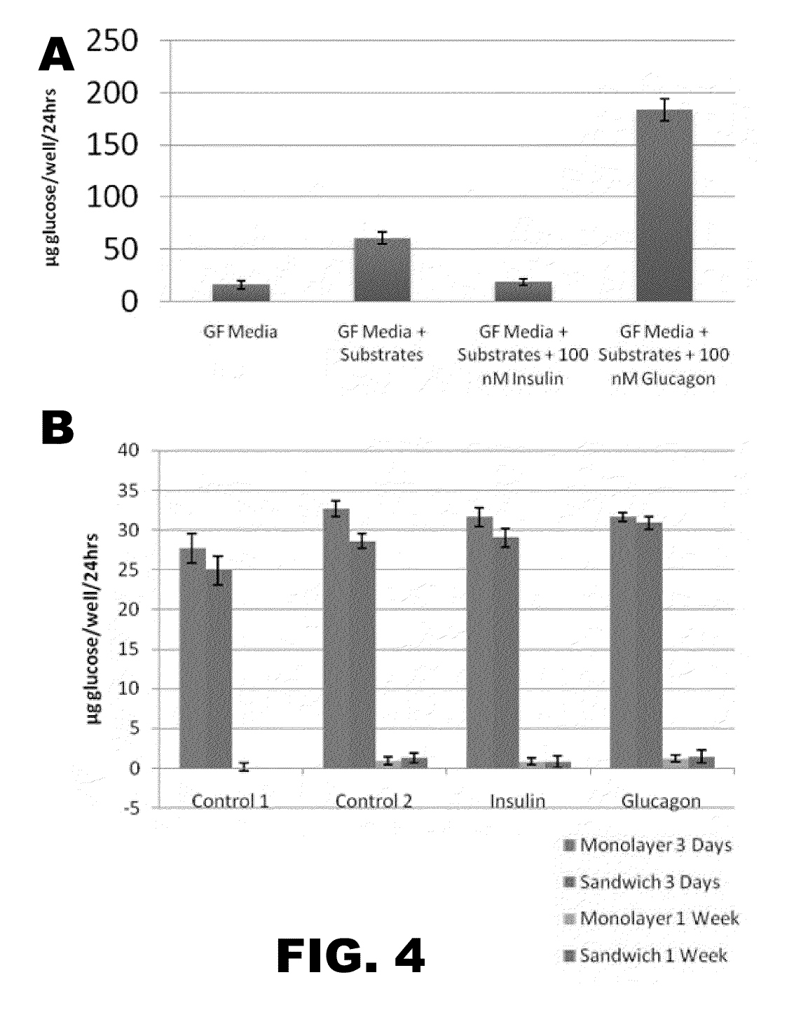

Cells were also tested under the glycogen depletion conditions as described above, for the effect of hormones on glucose production. The MPCCs were subjected to glycogen depletion for 12 hours, followed by stimulation of gluconeogenesis using lactate or pyruvate, in the presence or absence of insulin, glucagon or dexamethasone. Released glucose was measured 24 hours later. FIG. 3 summarizes the results as a bar graph of glucose production (μM) at 24 hours in the MPCCs, showing normal hormonal responsiveness in the MPCCs. FIG. 4 is a pair of bar graphs comparing gluconeogenesis (pg glucose/well/24 hours) after 1 week in human hepatocytes maintained in micropatterned co-culture with 3T3-J2 murine embryonic fibroblasts in glucose free medium and in the presence or absence of insulin or glucagon (upper panel), and in human hepatocytes maintained in conventional monoculture in the presence or absence of insulin and glucagon as indicated.

### Example 2

### Albumin and Urea Secretion by Hepatocytes in Conventional Culture and MPCC

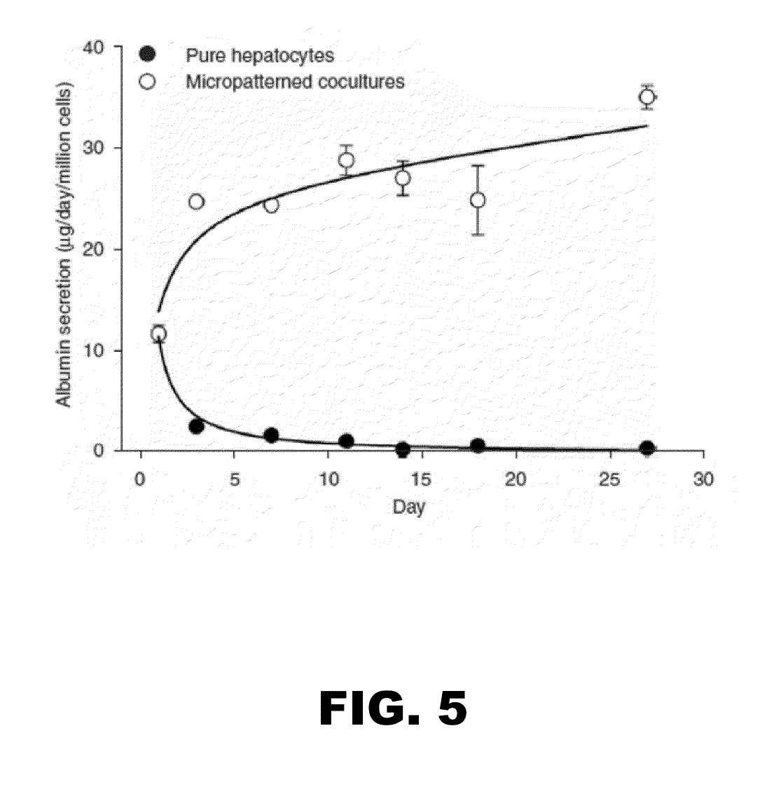

Albumin and urea secretion of hepatocytes in conventional (mono) culture and in MPCCs were compared by ELISA measurement of albumin secretion and a colorimetric assay of urea and albumin secretion, according to standard technique and protocol. FIGS. 5-9 show the results. In short, urea concentration was assayed using a colorimetric endpoint assay using diacetylmonoxime with acid and heat (Stanbio Labs). Albumin content was measured using enzyme-linked immunosorbent assays (MP Biomedicals) with horseradish peroxidase detection and 3,3′,5,5′-tetramethylbenzidine (TMB, Fitzgerald Industries) as a substrate

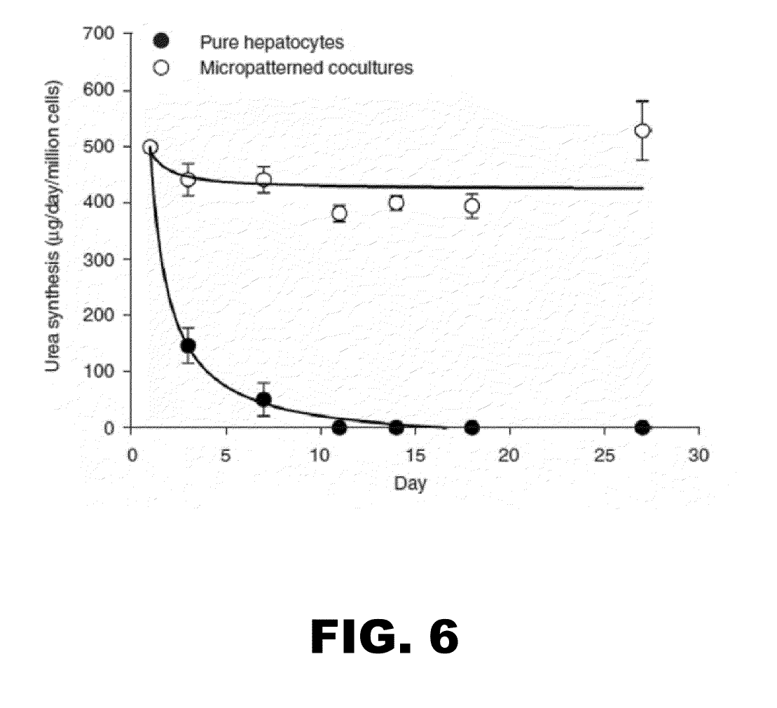

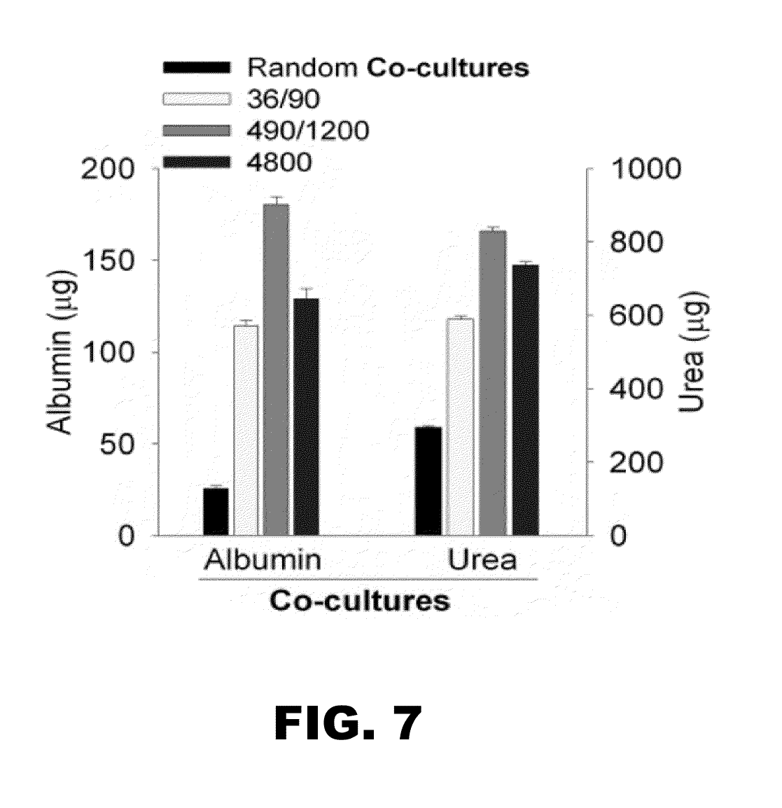

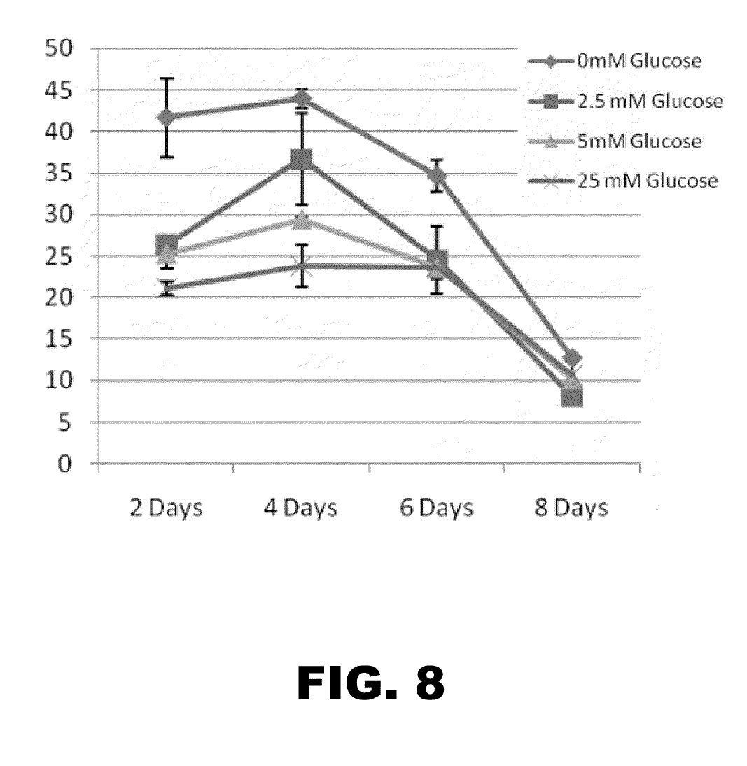

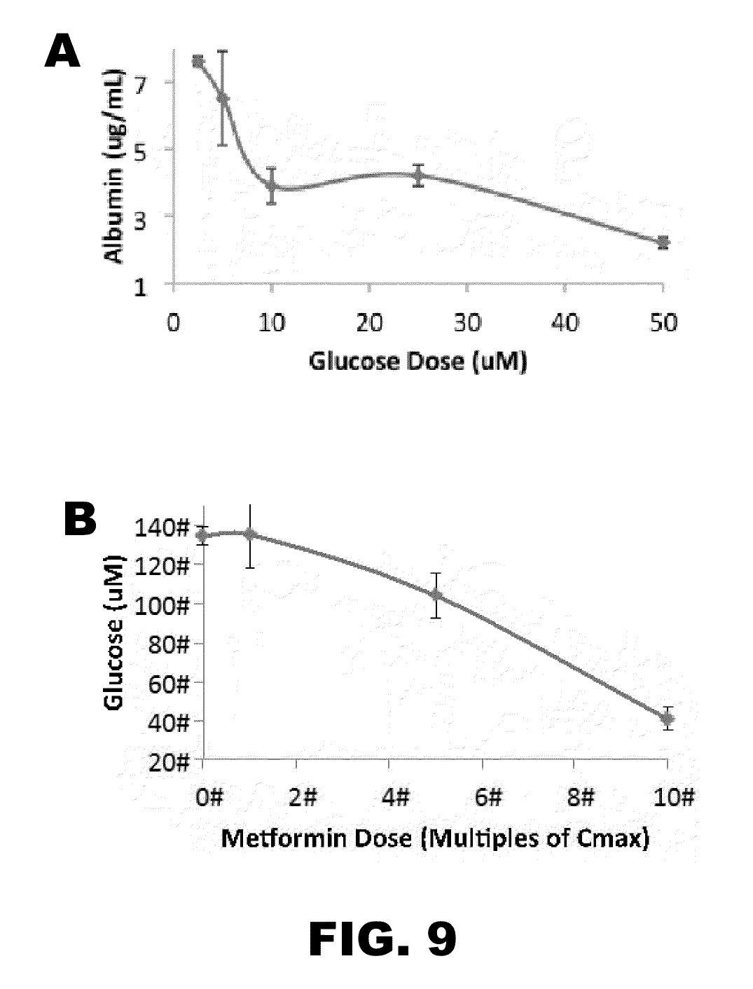

FIG. 5 is a graph of albumin secretion rate (pg/day/million cells) over time (days) in a population of pure (isolated) primary human hepatocytes in a conventional culture system (solid circles) and in a population of primary human hepatocytes in a micropatterned co-culture with 3T3-H2 murine embryonic fibroblast cells (open circles). MPCC hepatocytes achieved and maintained high levels of albumin secretion up to 27 days (longest period measured), while albumin secretion in hepatocytes in conventional culture rapidly dropped within 3 days to very low levels and was undetectable after about 14 days. FIG. 6 is a graph of urea synthesis rate (pg/day/million cells) over time (days) in a population of pure (isolated) primary human hepatocytes in a conventional culture system (solid circles) and in a population of primary human hepatocytes in a micropatterned co-culture with 3T3-H2 murine embryonic fibroblast cells (open circles). MPCC hepatocytes achieved and maintained high levels of urea synthesis up to 27 days, while albumin secretion in hepatocytes in conventional culture rapidly dropped within 3 days to very low levels and was undetectable after about 12 days. FIG. 7 is a bar graph comparing cumulative albumin secretion (μg; at left of graph) and urea synthesis (μg; at right of graph) over two weeks of populations of primary human hepatocytes in either a random co-culture, or in a micropatterned co-culture, with 3T3-H2 murine embryonic fibroblast cells, where varying diameters of the hepatocyte islands and varying edge-to-edge distances among the islands were used, while total cell numbers/ratios were held constant. For example, “36/90” indicates a micropattern defined by 36 μm diameter islands, with 90 μm edge-to-edge spacing. At two weeks, significantly higher cumulative albumin secretion and urea synthesis was observed in MPCC hepatocytes as compared to cells in random co-culture. Urea production and albumin secretion in MPCC hepatocytes showed normal responsiveness to the presence of glucose and other agents. FIG. 8 is a graph of urea production in human hepatocytes maintained in micropatterned co-culture with 3T3-J2 murine embryonic fibroblasts, using a “500/1200” micropattern, for a period of 8 days, in the presence of glucose amounts varying from 0 mM (diamonds) to 25 mM (x′s). Urea production in the MPCC hepatocytes showed normal response to glucose. FIG. 9, upper panel, is a graph of albumin secretion in human hepatocytes maintained in micropatterned co-culture with 3T3-J2 murine embryonic fibroblasts, in the presence of varying levels of glucose from 0 μM to 50 μM, showing down regulation of albumin secretion at high levels of glucose; lower panel is a graph of glucose production at 24 hours, and following administration of a varying dose of metformin, in human hepatocytes maintained in micropatterned co-culture with 3T3-J2 murine embryonic fibroblasts, showing dose-dependent blockage by metformin of gluconeogenesis.

### Example 3

### Morphology and Glycogen Lysis and Synthesis

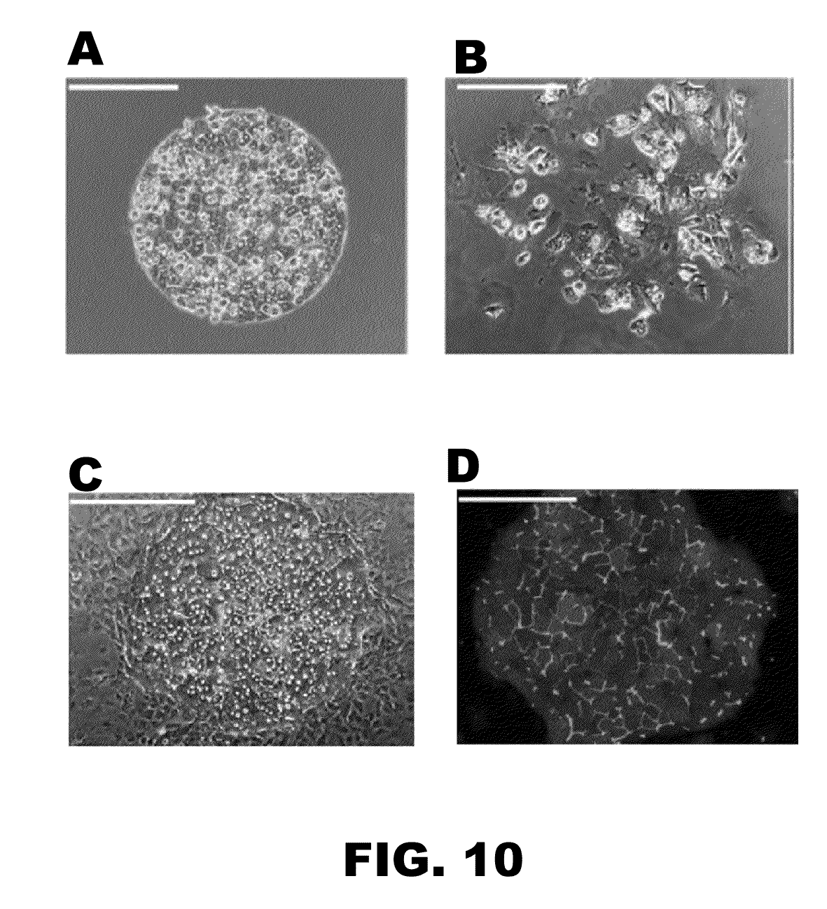

Cellular morphology of hepatocytes in conventional (mono) culture and MPCCs were also compared. FIGS. 10A-D is a series of photomicrographs of hepatocytes after varying periods and varying culture conditions. FIG. 10A shows pure hepatocytes under conventional culture conditions after 1 day. FIG. 10B is a photomicrograph of pure hepatocytes under conventional culture conditions after 1 week, showing clear loss of normal, rounded morphology. In contrast, FIG. 10C is a photomicrograph of hepatocytes after 1 week in micropatterned co-culture with 3T3-J2 murine embryonic fibroblasts, showing cells maintaining a normal, rounded morphology. FIG. 10D is a photomicrograph of hepatocytes after 2 weeks in micropatterned co-culture with 3T3-J2 murine embryonic fibroblasts, showing transport of neurometric dye by transporters into the bile canaliculi between hepatocytes.

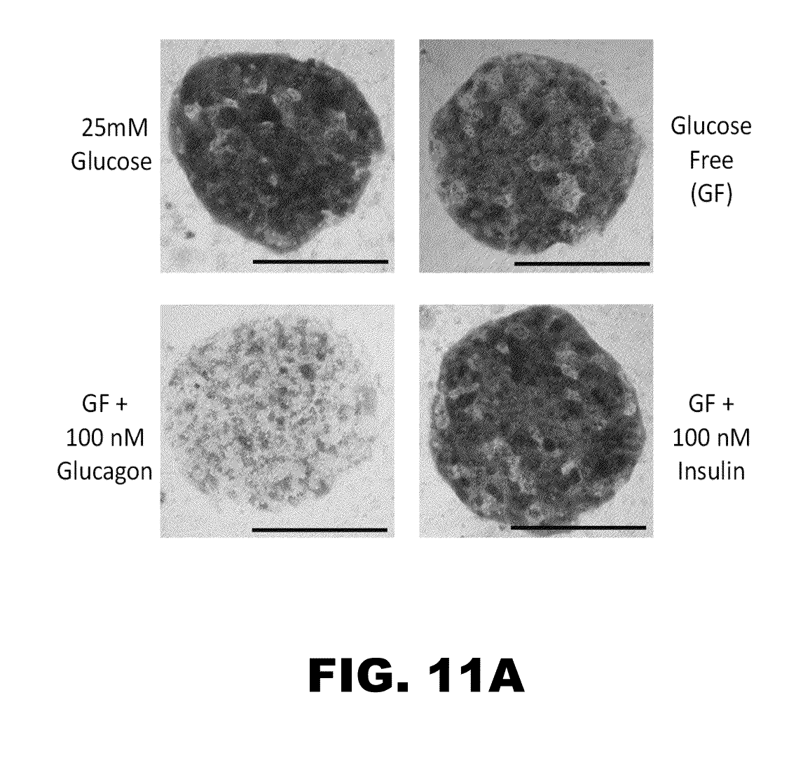

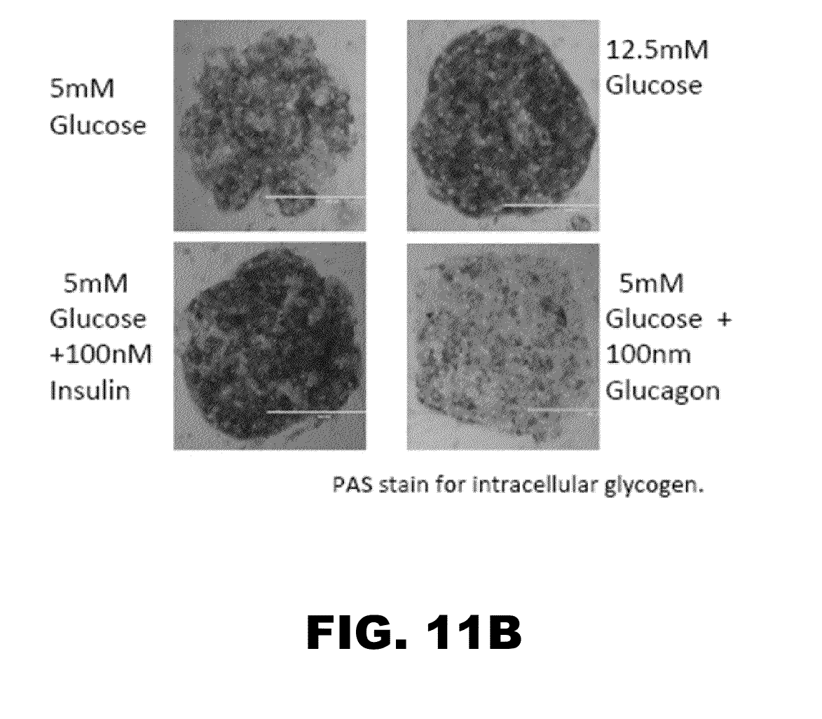

Glycogen lysis and glycogen synthesis in MPCC hepatocytes was also studied. The rate of glycogen lysis in MPCCs was measured by incubating cultures with glucose-free/hormone-free medium for 48 hours (fasting), followed by fixation of cultures using 4% paraformaldehyde and staining of intracellular glycogen (periodic acid Schiff stain (“PAS stain”)). The rate of glycogen synthesis was measured by first depleting glycogen stores as described above, followed by addition of glucose to medium (feeding) and staining of glycogen at specific time-points. Results at 24 hours are shown in FIGS. 11A-B. FIG. 11A is a panel of four photomicrographs of human hepatocytes maintained in micropatterned co-culture with 3T3-J2 murine embryonic fibroblasts, indicating glycogenolysis in the cells under four conditions: 25 mM glucose, glucose free, glucose free+100 nm glucagon, and glucose free+100 nm insulin, 24 hrs after treatment. The images indicate that MPCCs lysed glycogen when glucose was removed from media and at a higher rate when stimulated by glucagon. MPCCs continued storing glycogen when glucose was removed if stimulated by insulin. MPCCs were cultured in glucose free media for 2 days and depleted of all glycogen prior to the addition of 5 mM glucose. FIG. 11B is a panel of four photomicrographs of human hepatocytes maintained in micropatterned co-culture with 3T3-J2 murine embryonic fibroblasts, indicating via PAS stain for intracellular glycogen, glycogen synthesis in the cells under four conditions: 5 mM glucose, 12.5 mM glucose, 5 mM glucose+100 nm insulin, and 5 mM glucose+100 nm glucagon, 24 hours after treatment. MPCCs synthesized glycogen when placed in 5 mM glucose and at a higher rate when stimulated by insulin. MPCCs did not synthesize glycogen when placed in 5 mM glucose if stimulated by glucagon. Both glycogen lysis and glycogen synthesis by MPCC hepatocytes showed substantial and sustained levels of glycogen lysis and glycogen synthesis, and normal responsiveness to insulin and glucagon stimulation.

### Example 4

### Enzyme Expression

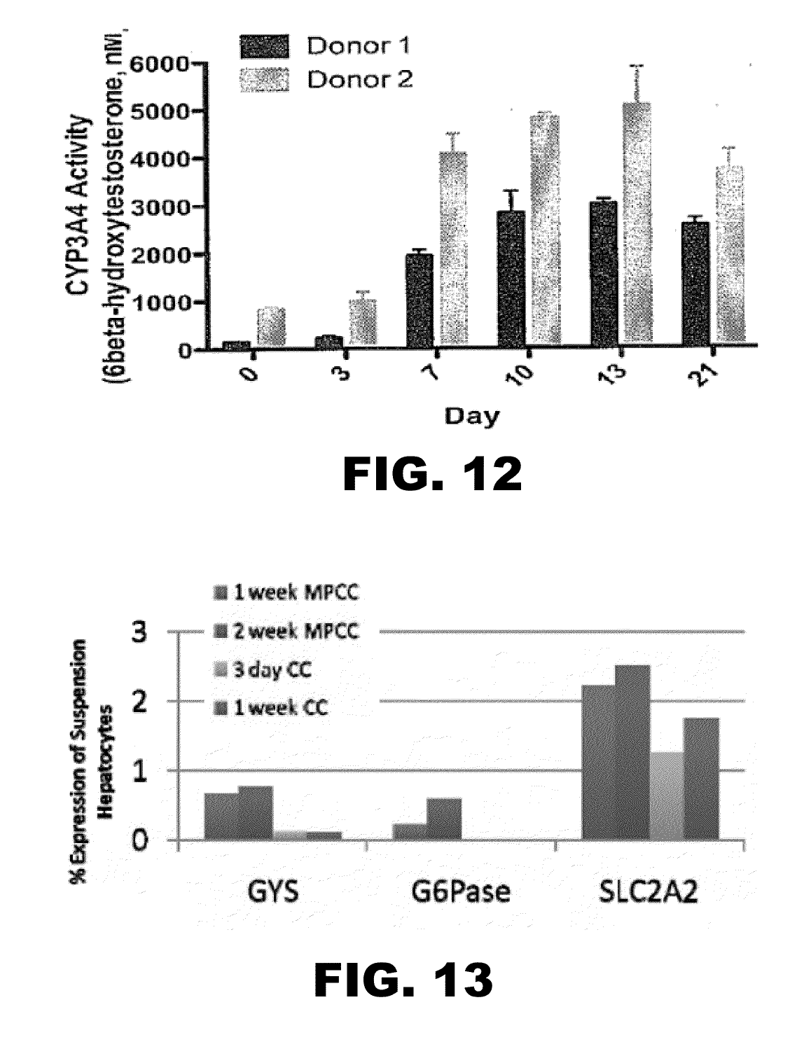

Expression of genes encoding enzymes involved in glucose pathways in the liver was studied using standard RT-qPCR technique. Results are shown in FIGS. 12-14. FIG. 12 is bar graph comparing CYP3A4 activity in hepatocytes from two human donors, maintained in micropatterned co-culture with 3T3-J2 murine embryonic fibroblasts, over a period of 0-21 days, showing sustained CYP3A4 expression over 21 days (longest period studied). FIG. 13 is a bar graph of RT-qPCR levels normalized to expression level in suspension hepatocytes, of glycogen synthase (GYS), glucose-6-phosphatase (G6Pase), and glucose transporter 2 (SL1A2), following 1 or 2 weeks in micropatterned co-culture with 3T3-J2 murine embryonic fibroblasts, or 3 days or 1 week in conventional (mono) culture.

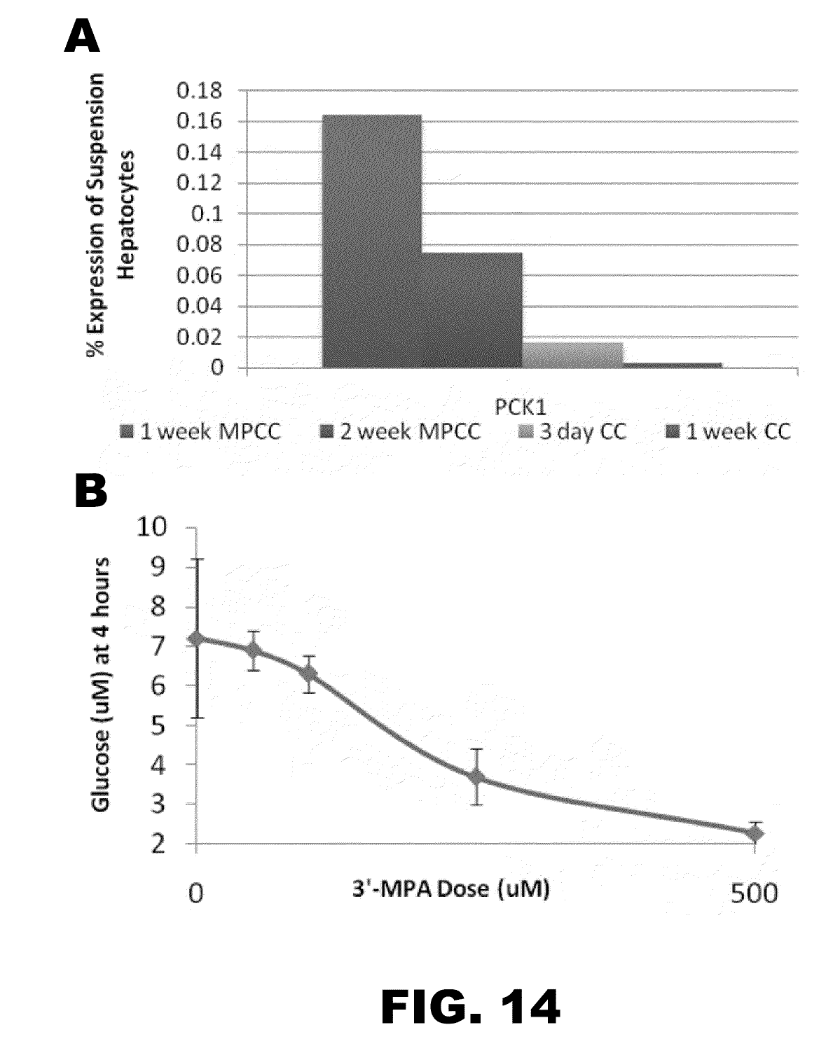

FIG. 14, upper panel, is a bar graph of expression level, as RT-qPCR level normalized to expression level in suspension hepatocytes, of phosphoenolpyruvate carboxykinase 1 (PCK1), following 1 or 2 weeks in micropatterned co-culture with 3T3-J2 murine embryonic fibroblasts, or 3 days or 1 week in conventional (mono) culture, showing higher levels of PCK1 expression in MPCC hepatocytes which were sustained at least to two weeks (longest period measured).

### Example 5

### Hepatocyte Steatosis

Fat metabolism in the MPCC hepatocytes was also examined to determine whether cells exhibited normal retention of lipids and to rule out possible impairment of the normal processes of synthesis and elimination of triglyceride fat. High glucose tends to cause mitochondrial impairment which shuts down beta-oxidation (breakdown of fatty acids into acetyl-coA that feeds into Kreb's cycle for ATP production) and generates fatty acids build up (i.e. lipids).

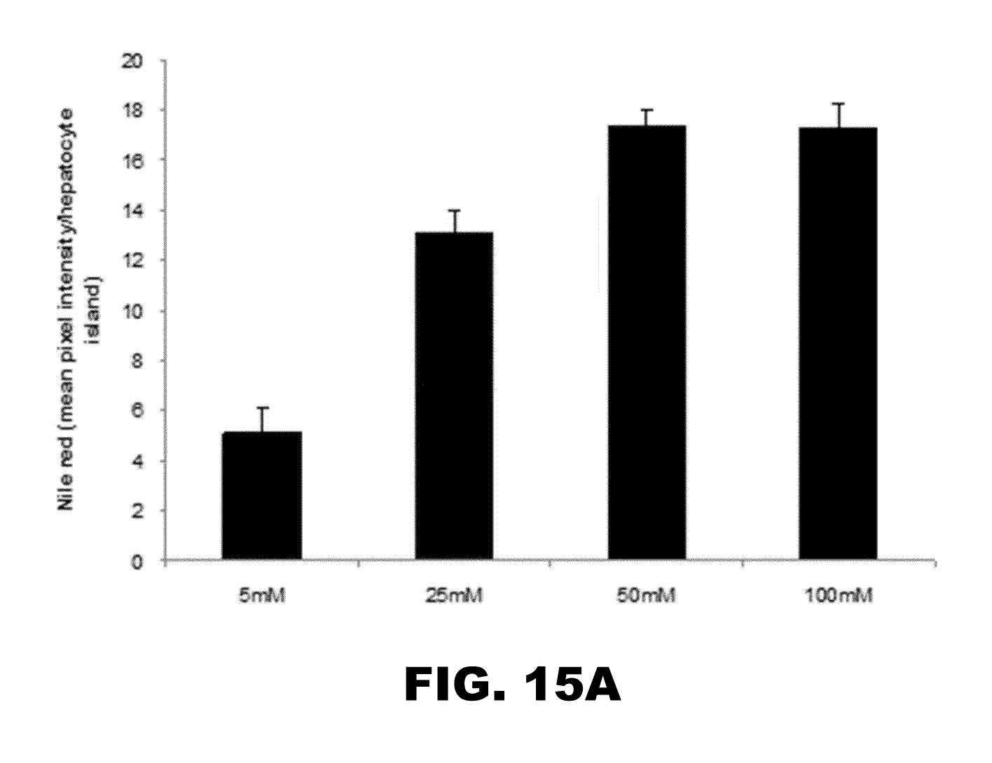

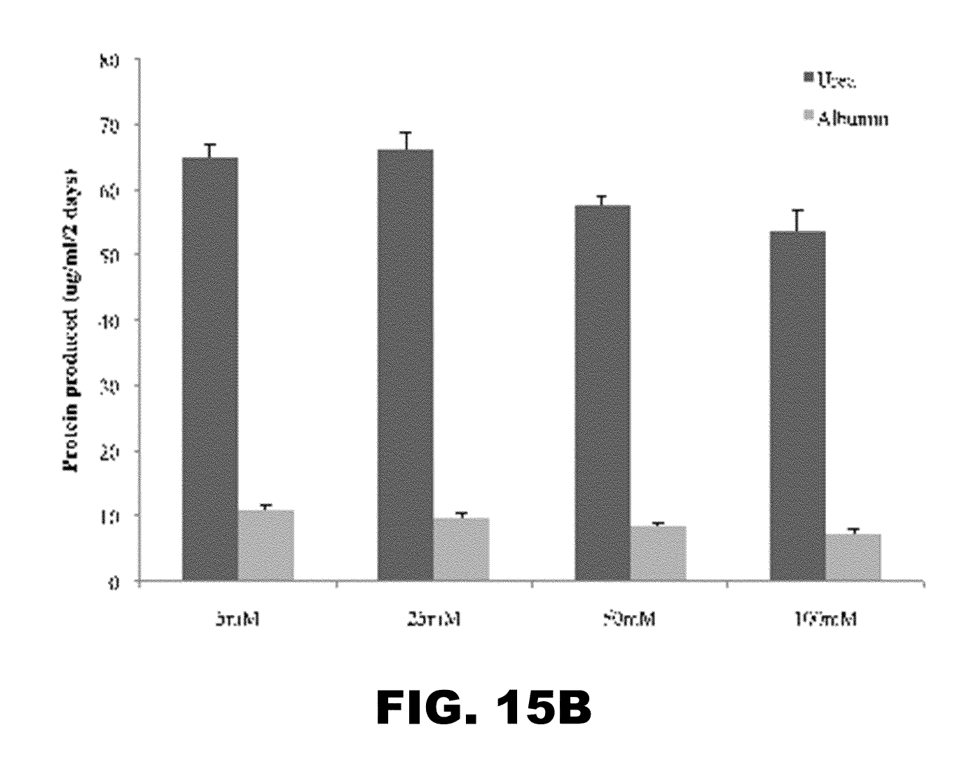

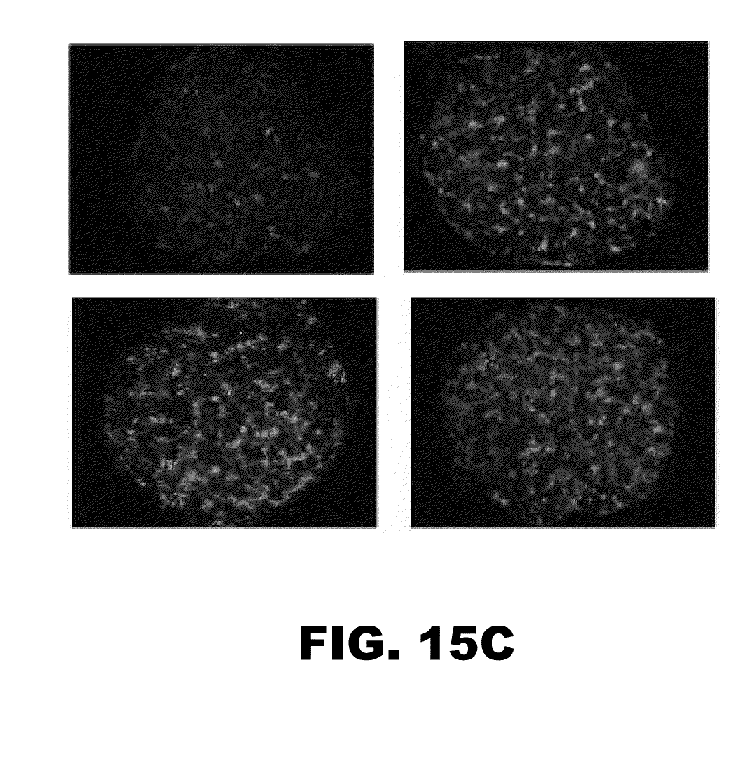

Intra-cellular lipids were measured via Nile red following exposure of the cells to varying levels of glucose (FIG. 15). Cells were exposed to glucose for 6 days. FIG. 15A is a bar graph of amount of fluorescence (mean fluorescence/island) from lipid staining using Nile red (FIG. 15C) or nuclei staining using DAPI, in human hepatocytes maintained in micropatterned co-culture with 3T3-J2 murine embryonic fibroblasts, indicating hepatocyte steatosis as a measure of glucose toxicity.

### Example 6

### Reuse of MPCCs

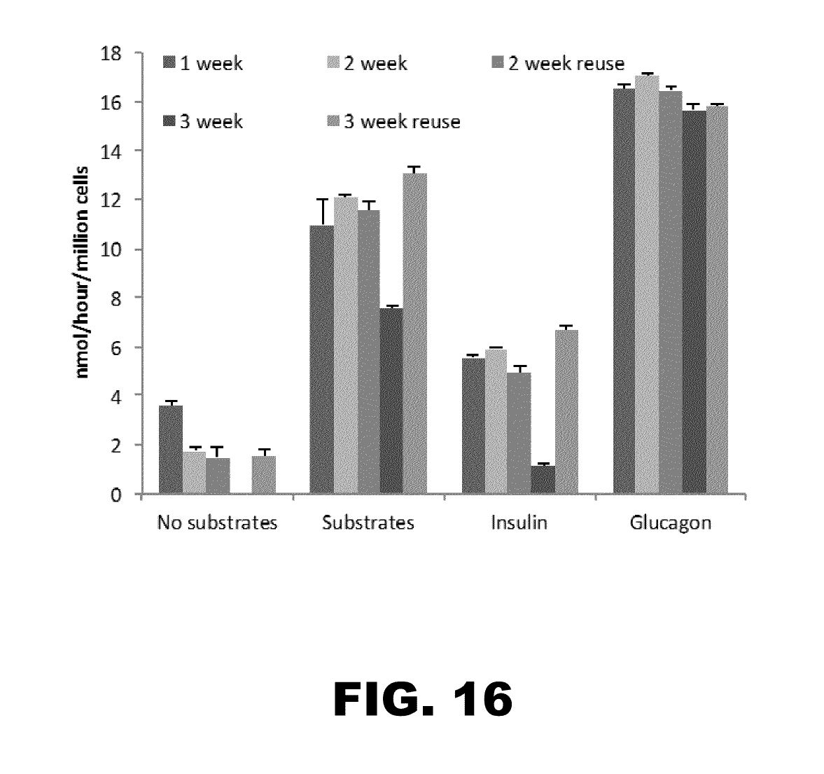

MPCCs were evaluated for robustness by examining gluconeogenesis and hormonal responsiveness, as described above, in MPCCs used once previously. For example, gluconeogenesis and hormonal responsiveness was measured a first time in an MPCC at week 1 and then a second time at week 2. FIG. 16 is a bar graph of gluconeogenesis rate (nmol/hour/million cells) in human hepatocytes maintained in micropatterned co-culture with 3T3-J2 murine embryonic fibroblasts, under varying conditions and at varying time periods including re-use of the co-culture. “2 week reuse” refers to co-cultures probed for gluconeogenesis and hormonal responsiveness (with glucagon or insulin) on week 1 and then reused on week 2. “3 week reuse” refers to co-cultures probed for gluconeogenesis on week 2, and reused on week 3. For comparison, bars labeled “1 Week”, “2 week” or “3 week” indicate data from co-cultures probed for gluconeogenesis for the first time at the time point indicated. The data show that even after doing a 72 hour perturbation for assessing gluconeogenesis (in glucose free medium), the cultures recover such that levels of gluconeogensis measured a second time one week later are as good, if not better (i.e., week 3 reuse vs. week 3), than naive cultures. The results suggest that 1) MPCCs can be used to study recovery of liver functions following feeding and starvation conditions configured to mimic clinical scenarios; and 2) MPCCs can be reliably used repeatedly, thereby improving efficiency of data collection, for example in a drug development program.

### Example 7

### Comparative Functional Characterization of MPCCs and Monoculture

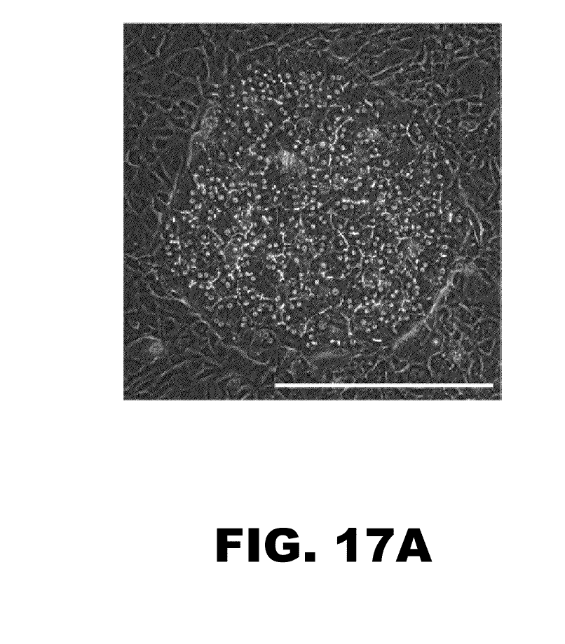

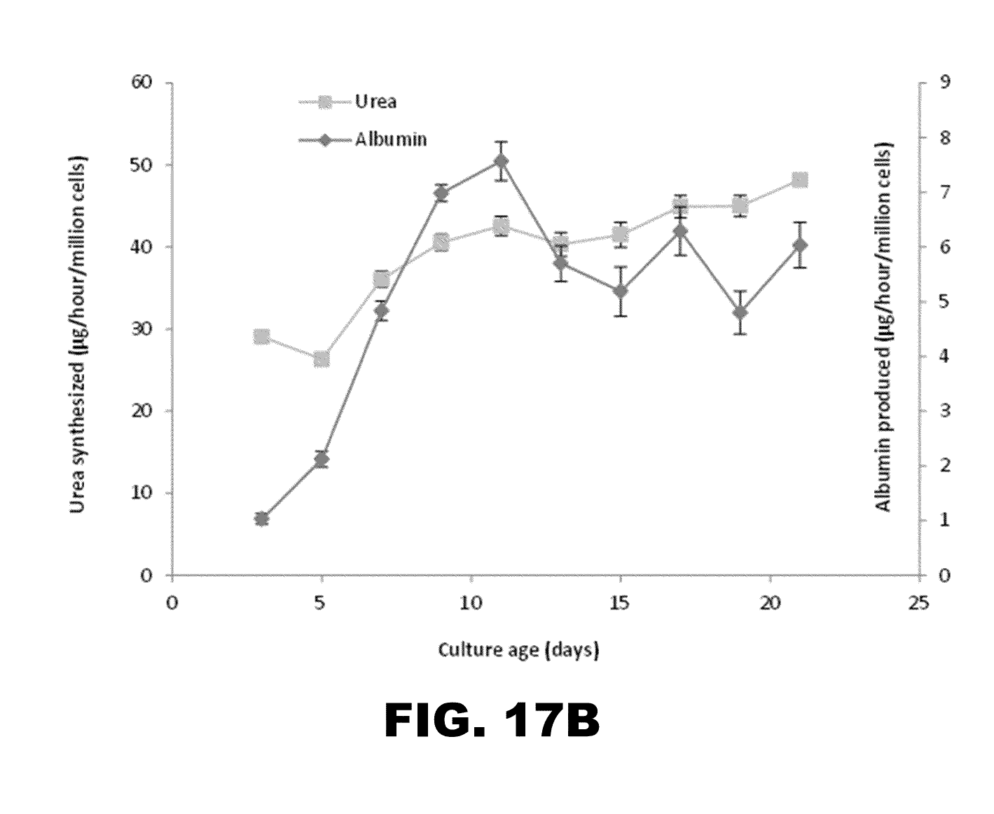

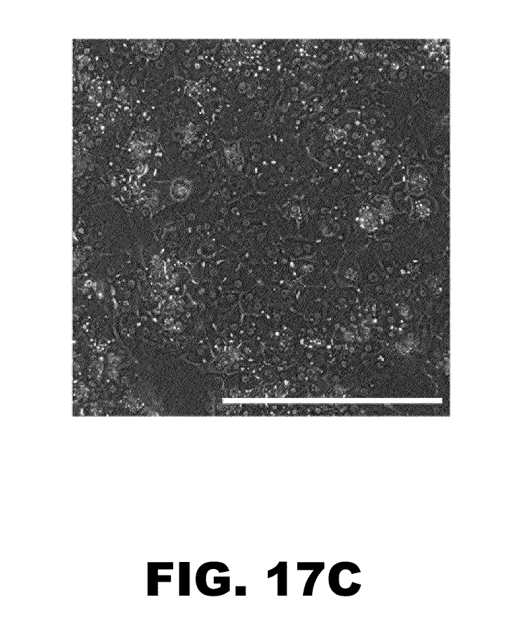

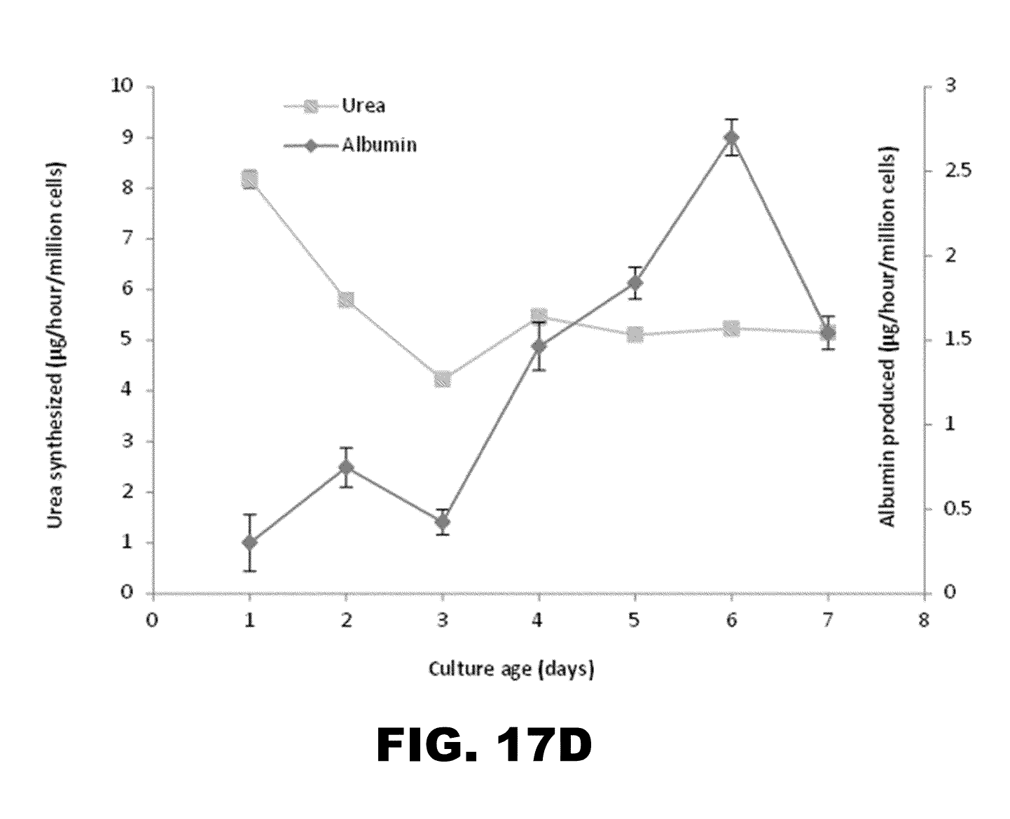

FIGS. 17A and C are photomicrographs each showing morphology of hepatocytes in MPCC (A), and in monolayer culture (C), both representative images from day 7. FIGS. 17B and D are graphs of rate of urea synthesis and albumin production in hepatocytes over 3 weeks in MPCC (B) and in monolayer culture (D). The results indicate that when it comes to other liver functions, conventional cultures (panels C and D) also demonstrate those functions, albeit at lower levels (on a per cell basis) than micropatterned co-cultures (panel A and B). However, as shown in FIG. 4 in Example 1, conventional cultures show little to no hormonal responsiveness, and even basal glucose production drops off sharply. Thus, in comparison to conventional culture, over time MPCCs demonstrate comparable or improved urea synthesis and albumin production in hepatocytes, while in conventional cultures hormonal responsiveness and basal glucose production disproportionately decline when compared to other major liver functions such as albumin production and urea synthesis. Thus the data indicate that micropatterned co-cultures can be used for a large repertoire of applications for which conventional cultures are currently used, and may be more reliable.

What is claimed is:

1. A composition comprising a population of hepatocytes and at least one non-parenchymal cell population in co-culture, wherein the hepatocytes are obtained from one or more human donors suffering from a metabolic disorder of the liver.

2. The composition of claim 1, wherein the metabolic disorder of the liver is selected from including Type-2 diabetes, metabolic syndrome, non-alcoholic fatty liver disease (NAFLD), non-alcoholic steatohepatitis (NASH) and cardiovascular disease.

3. The composition of claim 1, wherein the metabolic disorder of the liver is Type 2 diabetes.

4. The composition of claim 1, wherein the population of hepatocytes demonstrates, relative to donor hepatocytes obtained from donors not suffering from a metabolic disorder of the liver, increased rate of basal gluconeogenesis.

5. The composition of claim 1, wherein the population of hepatocytes demonstrates, is relative to donor hepatocytes obtained from not suffering from a metabolic disorder of the liver, increased rate of glucagon-induced gluconeogenesis.

6. The composition of claim 1, further comprising a culture substrate, wherein the population of hepatocytes are disposed in a micropattern on the culture substrate.

7. The composition of claim 1, wherein the population of hepatocytes and the at least one non-parenchymal cell population are maintained in vitro for at least three days.

8. The composition of claim 1, wherein the population of hepatocytes and the at least one non-parenchymal cell population are maintained in vitro for at least seven days.

9. The composition of claim 1, wherein the population of hepatocytes and the at least one non-parenchymal cell population are maintained in vitro for at least 27 days.

10. The composition of claim 1, wherein at least one of the non-parenchymal cell populations comprises stromal cells.

11. The composition of claim 10, wherein the stromal cells are selected from the group consisting of fibroblasts, fibroblast-derived cells, macrophages, endothelial cells, stellate cells, pericytes, inflammatory cells, cholangiocytes and other types of stromal cells, and combinations thereof.

12. The composition of claim 11, wherein the stromal cells comprise embryonic fibroblasts.

13. The composition of claim 12, wherein the stromal cells comprise murine embryonic fibroblasts.

14. The composition of claim 13, wherein the stromal cells comprise 3T3-J2 murine is embryonic fibroblasts.

15. The composition of claim 1, wherein at least one non-parenchymal cell population is obtained from an individual suffering from a metabolic disorder of the liver.

16. The composition of claim 6, wherein the culture substrate comprises a glass surface, a polystyrene surface, or a silicon surface.

17. The composition of claim 6, further comprising a biopolymer scaffold disposed on the culture substrate.

18. The composition of claim 6, wherein the micropattern comprises a predetermined two-dimensional pattern of multiple microdots, the micropattern defined by a microdot diameter and an edge-to-edge spacing between each of any two neighboring microdots.

19. The composition of claim 18, wherein each microdot has a diameter of 10 μm to 1000 μm.

20. The composition of claim 18, wherein the edge-to-edge spacing between each microdot is about 200 μm to about 1000 μm.

21. The composition of claim 18, further comprising a cell adhesion molecule applied to the culture substrate at the microdots, wherein the cell adhesion molecule is a material to which the hepatocytes selectively adhere relative to inter-microdot space.

22. The composition of claim 21 wherein the non-parenchymal cell population selectively occupies inter-microdot space.

23. The composition of claim 21, wherein the cell adhesion molecule is selected from the group consisting of collagen, fibronectin, vitronectin, laminin, Arg-Gly-Asp (RGD) peptide, Tyr-Ile-Gly-Ser-Arg (YIGSR) peptide, glycosaminoglycans, hyaluronic acid, integrins, ICAMs, selectins, cadherins and cell surface protein-specific antibodies.
